(19) United States
(12) Patent Application Publication
(10) Pub. No.: US 2025/0017961 A1
(43) Pub. Date: Jan. 16, 2025

(54) T CELL IMMUNOTHERAPY DERIVED FROM HIGHLY FUNCTIONAL AUTOLOGOUS STEM CELL MEMORY T CELLS

(71) Applicants: UNIVERSITE PARIS-SACLAY, GIF SUR YVETTE (FR); ASSISTANCE PUBLIQUE - HÔPITAUX DE PARIS, PARIS (FR); INSERM (INSTITUT NATIONAL DE LA SANTÉ ET DE LA RECHERCHE MÉDICALE), PARIS (FR); INSTITUT GUSTAVE ROUSSY, VILLEJUIF (FR)

(72) Inventors: YASSINE TAOUFIK, PARIS (FR); MARIE-GHISLAINE MADELEINE DE GOËR DE HERVE, LE PLESSIS-ROBINSON (FR)

(21) Appl. No.: 18/704,487

(22) PCT Filed: Oct. 26, 2022
(86) PCT No.: PCT/EP2022/080008

§ 371 Date: Apr. 25, 2024

(30) Foreign Application Priority Data
Oct. 26, 2021 (EP) 21306493.4

(51) Int. Cl. A61K 35/17; A61K 39/00; C12N 5/0783

(52) U.S. Cl. CPC A61K 35/17; A61K 39/4611; A61K 39/4644; A61K 39/464838; C12N 5/0636; C12N 2501/2307; C12N 2501/2315

(57) ABSTRACT

The present invention relates to a new and specific T-cell therapy strategy based on the use of memory stem T-cells (Tscm), in particular highly functional memory stem T-cells (Tscm) negatively selected on the basis of inhibitory receptor expression. This new cellular immunotherapy may be applied to PML patients but also to other infections or cancers for which the specific memory T cell responses are functionally impaired.

### FIELD OF THE INVENTION

The present invention relates to the field of medicine, in particular to the treatment of a cancer or a pathogen-caused disease, preferably a disease caused by a human polyomavirus such as Progressive Multifocal Leukoencephalitis, using a T cell immunotherapy.

### BACKGROUND OF THE INVENTION

Progressive Multifocal Leukoencephalitis (PML) is a demyelinating, opportunistic disease with a poor prognosis, associated with the replication of the polyomavirus JC (JCV) in the central nervous system. PML is observed exclusively during a prolonged and severe cellular immunosuppression, mainly in AIDS patients or patients with malignant hemopathies, or following immunosuppressive therapies, including the new potent immunosuppressive biotherapies. This devastating disease is associated with a high mortality and major neurological sequelae in survivors.

PML is related to an impaired intra-cerebral immune control of virus replication by cytotoxic memory CD8 T lymphocytes, which require functional memory CD4 T cells for their optimal functionality. Impairment of anti-JC virus CD8 T cells responses involve several mechanisms including anergy, and functional exhaustion with overexpression of inhibitory receptors such as PD1, LAG3, TIGIT, TIM3, CTLA4, CD160.

There is no specific antiviral treatment for PML. The only therapeutic approach that showed some efficacy is the functional recovery of anti-JCV T cell responses when it is possible, for instance by starting an effective antiretroviral treatment in HIV-infected patients or by stopping immunosuppressive therapies. However, such immune recovery may require a prolonged period during which JCV continues its replication, extending neurological lesions, and compromising the survival and the neurological prognosis.

Therefore, there is a strong need for new therapeutic options that can efficiently generate an effective and sustained immune response against JCV in PML patients.

### SUMMARY OF THE INVENTION

The inventors have developed a new and specific T-cell therapy strategy, based on the use of memory stem T-cells (Tscm), in particular highly functional Tscm negatively selected on the basis of inhibitory receptor expression. This approach is based on the key observation that in patients with severe and prolonged immunosuppression, such as PML patients, this rare memory T cell subset may maintain high functionality in terms of expansion and differentiation, and may generate ex vivo effective specific cytotoxic effectors against viral or tumoral antigens; while more differentiated memory T cell subsets such as effector memory (Tem) or central memory (Tcm), or effectors (Teff), are poorly functional against viral or tumoral antigens. This new cellular immunotherapy may be applied to PML patients but also to other infections or cancers for which the specific memory T cell responses are functionally impaired.

Accordingly, in a first aspect, the present invention relates to an in vitro method for obtaining a population of cells comprising antigen-specific T cells comprising
a) sorting from a cell sample from a subject suffering from a cancer or a pathogen-caused disease, in particular a cancer or a pathogen caused disease for which the specific memory T cell responses are functionally impaired, a population of Tscm cells having a cell surface phenotype comprising CD4+ or CD8+, CD45RA+, CCR7+ and/or CD62L+, and CD95+,
b) culturing said population of Tscm cells in the presence of antigen-presenting cells loaded with at least one antigen of interest or at least one peptide derived from at least one antigen of interest and, optionally, in the presence of IL-7 and IL-15 or other stimulatory cytokines, and optionally
c) recovering cells obtained in step b), in particular CD8+ and/or CD4+ cells, preferably CD8+ and CD4+ cells.

The population of Tscm cells sorted in step a) may have a cell surface phenotype further comprising PD1−, TIGIT−, LAG3−, TIM3−, CTLA4− and/or CD160−, preferably PD1−, TIGIT−, LAG3− and/or TIM3−. In particular, the population of Tscm cells sorted in step a) may have a cell surface phenotype further comprising PD1− and TIGIT−, and optionally LAG3−, TIM3−, CTLA4− and/or CD160−, preferably LAG3− and/or TIM3−, more preferably LAG3− and TIM3−.

Alternatively, the method may comprise
a) sorting from a cell sample from a subject a population of Tscm cells having a cell surface phenotype comprising CD4+ or CD8+, CD45RA+, CD95+, CCR7+ and/or CD62L+, PD1− and TIGIT−,
b) culturing said population of Tscm cells in the presence of antigen-presenting cells loaded with at least one immunogenic peptide derived from at least one antigen of interest and, optionally, in the presence of IL-7 and IL-15 or other stimulatory cytokines, and
c) sorting CD8+ cells, and optionally CD4+ cells, from the population of cells obtained in step b), preferably CD8+ cells and CD4+ cells from the population of cells obtained in step b).

Optionally, the population of Tscm cells sorted in step a) has a cell surface phenotype further comprising LAG3−, TIM3−, CTLA4− and/or CD160−, preferably LAG3− and/or TIM3−.

The population of Tscm cells sorted in step a) may have a cell surface phenotype further comprising CD3+, CD45RO−, CXCR3+ and/or CD122+, preferably CD3+ and CD45RO−.

In particular, the population of Tscm cells sorted in step a) may comprise cells having a cell surface phenotype comprising CD4+, CD8−, CD45RA+, CD95+, CCR7+, PD1− and TIGIT- and cells having a cell surface phenotype comprising CD4−, CD8+, CD45RA+, CD95+, CCR7+, PD1− and TIGIT−.

More particularly, the population of Tscm cells sorted in step a) may comprise cells having a cell surface phenotype comprising CD4+, CD8−, CD45RA+, CD95+, CCR7+, PD1−, TIGIT− LAG3 and TIM3−, preferably CD3+, CD45RO−, CD4+, CD8−, CD45RA+, CD95+, CCR7+, PD1−, TIGIT−, LAG3 and TIM3−, and cells having a cell surface phenotype comprising CD4−, CD8+, CD45RA+, CD95+, CCR7+, PD1−, TIGIT−, LAG3− and TIM3−, preferably CD3+, CD45RO−, CD4−, CD8+, CD45RA+, CD95+, CCR7+, PD1−, TIGIT−, LAG3− and TIM3−.

The antigen-presenting cells may be dendritic cells, monocytes, peripheral blood mononuclear cells (PBMCs), Epstein-Barr virus transformed B-lymphoblastoid cell line cells (EBV-BLCL cells), or artificial antigen presenting cells (AAPCs).

Preferably, the antigen-presenting cells are autologous to the subject.

Preferably, the antigen-presenting cells are monocytes or dendritic cells, more preferably monocytes or dendritic cells autologous to the subject.

Said at least one antigen of interest may be a pathogen antigen, preferably a viral, bacterial or fungal antigen, or an antigen expressed by tumor cells such as tumor-specific antigens (TSA) or tumor-associated antigens (TAA).

The subject may suffer from a cancer.

Said at least one antigen of interest may be an antigen expressed by tumor cells such as tumor-specific antigens (TSA) or tumor-associated antigens (TAA).

The subject may suffer from a disease caused by a human polyomavirus, preferably Progressive Multifocal Leukoencephalitis, Merkel cell carcinoma or BK virus associated nephropathy.

Preferably, said at least one antigen of interest is an antigen of a human polyomavirus, in particular selected from the group consisting of the polyomavirus JC, the polyomavirus MPCyV or the polyomavirus BK. More preferably, said at least one antigen of interest is an antigen of the polyomavirus JC or MPCyV, in particular an antigen of the polyomavirus JC.

In step b), the population of Tscm cells may be cultured in the presence of IL-7 and IL-15, and/or may be cultured for 8 to 20 days, preferably for 10 to 18 days, more preferably for 12 to 16 days.

The cell sample may be a bone marrow cell sample, a blood cell sample, a fractionated or unfractionated whole blood sample, a fractionated or unfractionated apheresis collection, tumor infiltrating lymphocytes, PBMCs, or a population enriched in T cells from a blood sample or PBMCs.

The present invention also relates to an isolated population of cells comprising antigen-specific CD8+ T cells, and optionally antigen-specific CD4+ T cells, obtained or obtainable by the method of the invention for obtaining a population of cells comprising antigen-specific T cells. The isolated population may comprise, or consists of, Tscm cells, T effector (Teff) cells, T central memory (Tcm) cells, and T effector memory (Tem) cells.

In the isolated population of cells of the invention, Tscm, Tcm and Tem cells may represent up to 90%, preferably from 50% to 90%, of the total cells and Teff cells may represent from 10% to 50%, preferably from 10% to 20%, of the total cells.

The present invention also relates to an in vitro method for obtaining a population of memory stem T-cells (Tscm cells) comprising sorting from a cell sample from a subject a population of Tscm cells having a cell surface phenotype comprising CD4+ or CD8+, CD45RA+, CD95+, CCR7+ and/or CD62L+, PD1− and TIGIT−, and optionally LAG3−, TIM3−, CTLA4− and/or CD160−, preferably LAG3− and/or TIM3−.

The subject may have a cancer or a pathogen-caused disease, in particular a cancer or a pathogen-caused disease for which the specific memory T cell responses are functionally impaired.

Preferably, the subject is suffering from a disease caused by a human polyomavirus.

More preferably, the subject is suffering from Progressive Multifocal Leukoencephalitis, Merkel cell carcinoma or BK virus associated nephropathy.

The present invention further relates to an isolated population of Tscm cells having a cell surface phenotype comprising (i) CD4+, CD45RA+, CD95+, CCR7+ and/or CD62L+, PD1−, TIGIT−, and optionally LAG3− and/or TIM3−, and/or (ii) CD8+, CD45RA+, CD95+, CCR7+ and/or CD62L+, PD1−, TIGIT−, and optionally LAG3−, TIM3−, CTLA4− and/or CD160−, preferably LAG3− and/or TIM3−, more preferably LAG3− and TIM3−.

The present invention also relates to an isolated population of cells comprising antigen-specific CD8+ T cells, and optionally antigen-specific CD4+ T cells, of the invention or an isolated population of Tscm cells of the invention as a cell therapy medicament. It also relates to a pharmaceutical composition comprising said isolated population of cells comprising antigen-specific CD8+ T cells of the invention or said isolated population of Tscm cells of the invention, and a pharmaceutically acceptable carrier and/or excipient.

The present invention further relates to said isolated population of cells comprising antigen-specific CD8+ T cells, and optionally antigen-specific CD4+ T cells, of the invention, said isolated population of Tscm cells of the invention or said pharmaceutical composition for use in the treatment of a cancer or a pathogen-caused disease, in particular a cancer or a pathogen-caused disease for which the specific memory T cell responses are functionally impaired.

Preferably, the disease to be treated is a disease caused by a polyomavirus, more preferably caused by a human polyomavirus. In particular, the disease to be treated may be Progressive Multifocal Leukoencephalitis, Merkel cell carcinoma or BK virus associated nephropathy.

Preferably, the disease to be treated is a pathogen-caused disease wherein the pathogen is the polyomavirus JC and the disease is Progressive Multifocal Leukoencephalitis (PML). Alternatively, the disease to be treated is a pathogen-caused disease wherein the pathogen is the polyomavirus MCPyV and the disease is Merkel cell carcinoma, or the disease to be treated is a pathogen-caused disease wherein the pathogen is the polyomavirus BKV and the disease is BK virus associated nephropathy.

Preferably, the cells used for the treatment are autologous to the subject to be treated.

The dose of isolated population of cells or pharmaceutical composition to be administered may comprise from 1000 to 10,000,000 antigen-specific CD8+ T cells/kg of body weight of the subject. The dose may further comprise from 1000 to 10,000,000 antigen-specific CD4+ T cells/kg of body weight of the subject.

The present invention further relates to the use of an isolated population of cells of the invention or a pharmaceutical composition of the invention for preparing a medicament for treating a cancer or a pathogen-caused disease, in particular a cancer or a pathogen-caused disease for which the specific memory T cell responses are functionally impaired.

The present invention further relates to a method for treating a subject suffering from a cancer or a pathogen-caused disease, in particular a cancer or a pathogen-caused disease for which the specific memory T cell responses are functionally impaired, comprising administering to said subject a therapeutically efficient amount of an isolated population of cells of the invention or a pharmaceutical composition of the invention.

### BRIEF DESCRIPTION OF THE FIGURES

FIG. 1: expression of any combination of inhibitory receptors (PD1, TIGIT, LAG3, TIM3) on Tscm, Tcm, Tem CD4 or CD8 T cells. Each line represents one patient.

FIG. 2: inhibitory receptors include mainly PD1 and/or TIGIT. Black parts of the pie chart represent cells positive for PD1 and/or TIGIT, alone or in combination with other inhibitory receptors. Grey parts represent cells positive for other inhibitory receptors than PD1 and/or TIGIT. White parts represent cells negative for inhibitory receptors. Each line represents one patient.

FIG. 3: Gating strategy for isolation of highly functional Tscm. The same gating strategy was applied to CD4 and CD8 T cells.

FIG. 4: Proliferation capacities of the different sorted T cell subsets. Left: Total Tscm (pooled CD4 and CD8 Tscm) versus more differentiated CD45RO+ memory cells (pooled CD4 and CD8 Tcm and Tem). Right: Tscm negative for PD1, TIGIT, TIM3 and LAG3 versus Tscm positive for PD1 and/or TIGIT and/or TIM3 and/or LAG3. The expansion rate was calculated as follows: [number of cells in the culture at day 14]/[number of cells at day 0]. Statistical significance: *p<0.05, wilcoxon test.

FIG. 5: Specific cytotoxicity potential of the cells obtained after 14 days of culture from the different sorted T cell subsets. Statistical significance: *p<0.05, wilcoxon test. The cytotoxic potential has been evaluated by the expression of Granzyme b and Perforin after re-stimulation by JCV-peptide loaded autologous cells (CD14 and CD3-depleted PBMC).

FIG. 6: Differentiation of PD1− TIGIT− Tscm CD8 T cells after 14-days in vitro culture.

### DETAILED DESCRIPTION OF THE INVENTION

The inventors have developed a new autologous and specific T-cell therapy strategy, to bypass the anti-JCV T-cell functional inhibition in PML patients. The approach is based on the use of memory stem T-cells (Tscm), preferably highly functional Tscm negatively selected on the basis of inhibitory receptor expression. Indeed, they observed that, in patients with severe and prolonged immunosuppression, such as PML patients, this rare memory T cell subset may maintain high functionality in terms of expansion and differentiation, and may generate ex vivo effective specific cytotoxic effectors against viral or tumoral antigens. They also demonstrated that in PML patients from different immunological backgrounds including HIV-infection, hematological malignancies and treatment with immunosuppressive biotherapies, Tscm cells express lower amounts of inhibitory receptors than more differentiated memory cells. They further demonstrated that a selection based on PD1 and TIGIT exclusion allows to deplete a major part of inhibitory receptors expressing Tscm. They also showed that these PD1− TIGIT− Tscm cells show better proliferation capacities and better cytotoxic capacities compared to PD1+ and/or TIGIT+ Tscm and to more differentiated memory cells. These cells differentiate efficiently in vitro to more differentiated memory cells including effector cells but a large part retain the Tscm phenotype allowing further cycles of differentiation in vivo after administration and therefore a prolonged therapeutic effect. After in vitro activation, differentiation and expansion, these cells are thus able to provide a population of cells comprising antigen-specific T cells and being able to generate an effective and sustained immune response against the JCV in PML patients. This new cell therapy-based personalized medicine can also be applied to other chronic viral infections or cancers for which the specific antiviral or anti-tumoral memory T cell responses are functionally impaired.

In a first aspect, the present invention relates to an in vitro method for obtaining a population of cells comprising antigen-specific T cells comprising
a) sorting from a cell sample from a subject a population of Tscm cells, i.e. a population of Tscm cells having a cell surface phenotype comprising (i) CD4+ or CD8+, (ii) CD45RA+, (iii) CD95+, and (iv) CCR7+ and/or CD62L+,
b) culturing said population of Tscm cells in the presence of antigen-presenting cells loaded with at least one antigen of interest or one or more peptides derived from said at least one antigen of interest, and, optionally, in the presence of IL-7 and IL-15 or other stimulatory cytokines, and optionally
c) recovering cells obtained in step b), in particular CD8+ and/or CD4+ cells, preferably CD8+ and CD4+ cells.

Preferably, in step a), the population of Tscm cells has a cell surface phenotype further comprising PD1−, TIGIT−, LAG3−, TIM3−, CTLA4− and/or CD160−, more preferably PD1−, TIGIT−, LAG3− and/or TIM3−.

In preferred embodiments, the method comprises
a) sorting from a cell sample from a subject a population of highly functional Tscm cells, i.e. a population of Tscm cells having a cell surface phenotype comprising (i) CD4+ or CD8+, (ii) CD45RA+, (iii) CD95+, (iv) CCR7+ and/or CD62L+, (v) PD1− and (vi) TIGIT−,
b) culturing said population of Tscm cells in the presence of antigen-presenting cells loaded with at least one antigen of interest or one or more peptides derived from said at least one antigen of interest, and, optionally, in the presence of IL-7 and IL-15 or other stimulatory cytokines, and optionally
c) recovering cells obtained in step b), in particular CD8+ and/or CD4+ cells, preferably CD8+ and CD4+ cells.

In particular, in step c), CD8+ cells, and optionally CD4+ cells, may be sorted from the population of cells obtained in step b).

Optionally, step a) may further comprise depleting cells expressing one or several other inhibitory receptors such as LAG3, TIM3, CTLA4 or CD160. In particular, step a) may further comprise depleting cells expressing LAG3, TIM3, CTLA4, and/or CD160. In this case, the population of sorted cells may have a cell surface phenotype comprising (i) CD4+ or CD8+, CD45RA+, CD95+, CCR7+ and/or CD62L+, PD1− and TIGIT− and (ii) LAG3−, TIM3−, CTLA4− and/or CD160−.

Preferably, step a) further comprises depleting cells expressing LAG3 and/or TIM3. In this case, the population of sorted cells may have a cell surface phenotype comprising (i) CD4+ or CD8+, CD45RA+, CD95+, CCR7+ and/or CD62L+, PD1− and TIGIT− and (ii) LAG3− and/or TIM3-, preferably a cell surface phenotype comprising CD4+ or CD8+, CD45RA+, CD95+, CCR7+ and/or CD62L+, PD1−, TIGIT−, LAG3− and TIM3−.

Optionally, step a) may further comprise depleting cells expressing CD45RO and/or selecting cells expressing CD3. In this case, the population of sorted cells may have a cell surface phenotype comprising (i) CD4+ or CD8+, CD3+, CD45RA+, CD45RO−, CD95+, CCR7+ and/or CD62L+, preferably CD4+ or CD8+, CD3+, CD45RA+, CD45RO−, CD95+, CCR7+ and/or CD62L+, PD1− and/or TIGIT−, more preferably CD4+ or CD8+, CD3+, CD45RA+, CD45RO−, CD95+, CCR7+ and/or CD62L+, PD1− and TIGIT−, and optionally (ii) LAG3−, TIM3−, CTLA4− and/or CD160−. Preferably, step a) further comprises depleting cells expressing LAG3 and/or TIM3, preferably depleting cells expressing LAG3 and TIM3. In this case, the population of sorted cells may have a cell surface phenotype comprising (i) CD4+ or CD8+, CD3+, CD45RA+, CD45RO−, CD95+, CCR7+ and/or CD62L+, preferably CD4+ or CD8+, CD3+, CD45RA+, CD45RO−, CD95+, CCR7+ and/or CD62L+, PD1− and/or TIGIT−, more preferably CD4+ or CD8+, CD3+, CD45RA+, CD45RO−, CD95+, CCR7+ and/or CD62L+, PD1− and TIGIT−, and (ii) LAG3− and/or TIM3−, preferably LAG3− and TIM3−.

As used herein, the term “CD4” refers to T-cell surface glycoprotein CD4, a glycoprotein that serves as a co-receptor for the T-cell receptor (TCR). In humans, the CD4 protein is encoded by the CD4 gene.

As used herein, the term “CD8” refers to a transmembrane glycoprotein that serves as a co-receptor for the T-cell receptor (TCR). There are two isoforms of the protein, alpha and beta, each encoded by a different gene. CD8 forms a dimer, consisting of a pair of CD8 chains. As used herein, the term “CD8” refers to the CD8-α chain encoded, in humans, by the CD8A gene.

As used herein, the term “CD3” refers to a protein complex and T cell co-receptor. In mammals, the complex contains a CD3γ chain, a CD3δ chain, and two CD3ε chains. The CD3 is part of a bigger complex which includes the T Cell Receptor (TCR). CD3 complex associated with the TCR is involved in the recognition of peptides bound to the major histocompatibility complex class I and II during the immune response. As used herein, the term “CD3” refers to the CD3γ chain encoded, in humans, by the CD3G gene, to the CD3δ chain encoded, in humans, by the CD3D gene or the CD3ε chain encoded, in humans, by the CD3ε gene.

As used herein, the term “CD45RA” refers to the 200- to 220-kDa isoform of the receptor-type tyrosine-protein phosphatase C also named CD45. In humans, the CD45 protein is encoded by the PTPRC gene. This tyrosine phosphatase is required for T-cell activation through the antigen receptor. The CD45RA isoform includes only the A protein region.

As used herein, the term “CD45RO” refers to the 180-kDa isoform of the receptor-type tyrosine-protein phosphatase C also named CD45. This isoform is the shortest CD45 isoform, which lacks all three of the A, B, and C regions.

As used herein, the term “CD95” refers to the Fas receptor, also known as Fas, FasR, apoptosis antigen 1 or tumor necrosis factor receptor superfamily member 6 (TNFRSF6). In humans, the CD95 protein is encoded by the FAS gene.

As used herein, the term “CCR7” refers to C—C chemokine receptor type 7, also known as CD197, and is a member of the G protein-coupled receptor family. In humans, the CCR7 protein is encoded by the CCR7 gene.

As used herein, the term “CD62L” refers to L-selectin, a calcium-dependent lectin that mediates cell adhesion by binding to glycoproteins on neighboring cells. In particular, CD62L mediates the adherence of lymphocytes to endothelial cells of high endothelial venules in peripheral lymph nodes In humans, the CD62L is encoded by the SELL gene.

As used herein, the term “PD1” refers to Programmed cell D protein 1 also known as CD279. PD1 is a cell surface receptor that belongs to the immunoglobulin superfamily and is expressed on the surface of T and B cells. In humans, the PD-1 protein is encoded by the PDCD1 gene.

As used herein, the term “TIGIT” refers to an immune receptor also known as T cell immunoreceptor with Ig and ITIM domains, WUCAM or Vstm3. In humans, the TIGIT protein is encoded by the TIGIT gene.

As used herein, the term “LAG3” refers to Lymphocyte-activation gene 3 also known as CD223. LAG3 is a cell surface molecule with diverse biologic effects on T cell function. In humans, the LAG3 protein is encoded by the LAG3 gene.

As used herein, the term “TIM3” refers to T cell immunoglobulin and mucin domain-containing protein 3 also known as Hepatitis A virus cellular receptor 2 (HAVCR2). TIM3 is a surface receptor implicated in modulating innate and adaptive immune responses. In humans, the TIM3 protein is encoded by the HAVCR2 gene.

As used herein, the term “CTLA4” refers to cytotoxic T-lymphocyte-associated protein 4 also known as CD152. CTLA4 is protein receptor that functions as an immune checkpoint and downregulates immune responses. In humans, the CTLA4 protein is encoded by the CTLA4 gene.

As used herein, the term “CD160” refers to a glycoprotein receptor on immune cells capable to deliver stimulatory or inhibitory signals that regulate cell activation and differentiation. In humans, the CD160 protein is encoded by the CD160 gene.

Additionally, the population of Tscm cells sorted in step a) may also express CXCR3 and/or CD122 and may be also selected on the basis of these additional markers. In this case, the population of sorted cells may have a cell surface phenotype comprising (i) CD4+ or CD8+, CD45RA+, CD95+, CCR7+ and/or CD62L+, CXCR3+ and/or CD122+, preferably CD4+ or CD8+, CD45RA+, CD95+, CCR7+ and/or CD62L+, CXCR3+ and/or CD122+, PD1− and/or TIGIT−, more preferably CD4+ or CD8+, CD45RA+, CD95+, CCR7+ and/or CD62L+, CXCR3+ and/or CD122+, PD1− and TIGIT−, or (ii) CD4+ or CD8+, CD3+, CD45RA+, CD45RO−, CD95+, CCR7+ and/or CD62L+, CXCR3+ and/or CD122+, preferably CD4+ or CD8+, CD3+, CD45RA+, CD45RO−, CD95+, CCR7+ and/or CD62L+, CXCR3+ and/or CD122+, PD1− and/or TIGIT−, more preferably CD4+ or CD8+, CD3+, CD45RA+, CD45RO−, CD95+, CCR7+ and/or CD62L+, CXCR3+ and/or CD122+, PD1− and TIGIT−. Optionally, these populations of sorted cells may have a cell surface phenotype further comprising LAG3−, TIM3−, CTLA4− and/or CD160−, preferably LAG3− and/or TIM3−, more preferably LAG3− and TIM3−.

As used herein, the term “CXCR3” refers to chemokine receptor CXCR3 also known as G protein-coupled receptor 9 (GPR9) and CD183. In humans, the CXCR3 is encoded by the CXCR3 gene. As used herein, the term “CD122” refers to Interleukin-2 receptor subunit beta also known as IL15RB. CD122 is a receptor for interleukin-2. This beta subunit is involved in receptor mediated endocytosis and transduces the mitogenic signals of IL2. In humans, the CD122 is encoded by the IL2RB gene.

As used herein, the term “cell surface phenotype” refers to the presence or absence of a combination of specific cell surface markers at the surface of the cells. By “cell surface marker” is intended a molecule expressed on the surface of a cell that can be detected, for example, using labeled antibodies or other means known in the art. A cell surface marker can comprise a protein, glycoprotein, or group of proteins and/or glycoproteins. In the present case, the population of Tscm cells sorted/selected in step a) may be identified by expression of a particular combination of markers comprising CD4 or CD8, CD45RA, CD95, CCR7 and/or CD62L, and preferably CD3, and the lack of expression of a particular combination of markers comprising PD1 and/or TIGIT, preferably PD1 and TIGIT, and optionally LAG3, TIM3, CTLA4 and/or CD160, preferably LAG3 and/or TIM3, more preferably LAG3 and TIM3. Optionally, the population of Tscm cells sorted/selected in step a) may be further identified by the lack of expression of CD45RO.

The population of Tscm cells obtained in step a) is enriched in Tscm cells having a particular cell surface phenotype.

In particular embodiments, the population of Tscm cells obtained in step a) is enriched in Tscm cells having a cell surface phenotype comprising CD4+ or CD8+, CD45RA+, CD95+, CCR7+ and/or CD62L+, and optionally CD3+, CD45RO−, CXCR3+ and/or CD122+, preferably in Tscm cells having a cell surface phenotype comprising CD4+ or CD8+, CD45RA+, CD95+, CCR7+ and/or CD62L+, CD3+ and CD45RO−. Preferably, the cell surface phenotype further comprises PD1−, TIGIT−, LAG3−, TIM3−, CTLA4− and/or CD160−, preferably PD1− and/or TIGIT− and optionally LAG3− and/or TIM3−, more preferably PD1− and TIGIT− and optionally LAG3− and/or TIM3−, and even more preferably PD1−, TIGIT−, LAG3− and TIM3−.

In preferred embodiments, the population of Tscm cells obtained in step a) is enriched in Tscm cells having a cell surface phenotype comprising CD4+ or CD8+, CD45RA+, CD95+, CCR7+ and/or CD62L+, PD1− and TIGIT−, and optionally LAG3−, TIM3−, CTLA4− and/or CD160−, preferably LAG3− and/or TIM3−, more preferably LAG3− and TIM3−. Preferably, Tscm cells have a cell surface phenotype further comprising CD3+ and CD45RO−.

By “enriched” is meant a composition comprising cells present in a greater percentage of total cells than is found in another composition. In particular, in the population obtained in step a), Tscm cells having a particular cell surface phenotype as defined above, e.g. a cell surface phenotype comprising CD4+ or CD8+, CD45RA+, CD95+, CCR7+ and/or CD62L+, PD1− and TIGIT−, and optionally LAG3−, TIM3−, CTLA4− and/or CD160−, preferably LAG3− and/or TIM3−, are present in a higher percentage of total cells as compared to their percentage in the cell sample. In the population obtained in step a), Tscm cells having said particular cell surface phenotype, e.g. a cell surface phenotype comprising CD4+ or CD8+, CD45RA+, CD95+, CCR7+ and/or CD62L+, PD1− and TIGIT−, and optionally LAG3−, TIM3−, CTLA4− and/or CD160−, preferably LAG3− and/or TIM3−, represent more than 70%, preferably represent more than 80%, 90%, 95% or 99% of the total cells in said population, even more preferably represent more than 95% or 99% of the total cells in said population.

Conversely, the population of Tscm cells obtained in step a) may be depleted in cells expressing inhibitory receptors PD1, TIGIT, LAG3, TIM3, CTLA4 and/or CD160, preferably in cells expressing inhibitory receptors PD1 and TIGIT, and optionally LAG3−, TIM3−, CTLA4− and/or CD160−, preferably LAG3 and/or TIM3. Preferably, the population of Tscm cells obtained in step a) is further depleted in cells expressing CD45RO. By “depleted” is meant a composition comprising cells present in a lower percentage of total cells than is found in another composition, in particular than is found in the cell sample. In preferred embodiments, in the population obtained in step a), Tscm cells having a cell surface phenotype comprising PD1+ and TIGIT+, and optionally LAG3+, TIM3+, CTLA4+ and/or CD160+, preferably LAG3+ and/or TIM3+, are present in a lower percentage of total cells as compared to their percentage in the cell sample. In particular, in the population obtained in step a), Tscm cells expressing inhibitory receptors PD1 and TIGIT, and optionally LAG3, TIM3, CTLA4 and/or CD160, preferably LAG3 and/or TIM3, may represent less than 5%, 2% or 1% of the total cells in said population.

Preferably, in the population obtained in step a), Tscm cells having a cell surface phenotype comprising CD4+ or CD8+, CD45RA+, CD95+, CCR7+ and/or CD62L+, preferably comprising CD4+ or CD8+, CD45RA+, CD95+, CCR7+ and/or CD62L+ and CD3+, more preferably comprising CD4+ or CD8+, CD45RA+, CD95+, CCR7+ and/or CD62L+, CD3+ and CD45RO−, represent more than 95%, 96%, 97%, 98% or 99% of the total cells in said population.

More preferably, in the population obtained in step a), Tscm cells having a cell surface phenotype comprising (i) CD4+ or CD8+, CD45RA+, CD95+, CCR7+ and/or CD62L+, PD1− and TIGIT−, preferably CD4+ or CD8+, CD45RA+, CD95+, CCR7+ and/or CD62L+, CD3+, PD1− and TIGIT−, more preferably CD4+ or CD8+, CD45RA+, CD95+, CCR7+ and/or CD62L+, CD3+, CD45RO−, PD1− and TIGIT−, and (ii) optionally LAG3−, TIM3−, CTLA4− and/or CD160−, preferably LAG3− and/or TIM3−, represent more than 95%, 96%, 97%, 98% or 99% of the total cells in said population. In this case, Tscm cells having a cell surface phenotype comprising CD4+ or CD8+, CD45RA+, CD95+, CCR7+ and/or CD62L+, PD1+ and/or TIGIT+, preferably CD4+ or CD8+, CD45RA+, CD95+, CCR7+ and/or CD62L+, CD3+, PD1+ and/or TIGIT+, more preferably CD4+ or CD8+, CD45RA+, CD95+, CCR7+ and/or CD62L+, CD3+, CD45RO−, PD1+ and TIGIT+, and optionally LAG3+, TIM3+, CTLA4+ and/or CD160+, preferably LAG3+ and/or TIM3+, may represent less than 5% of the total cells in the population, more preferably less than 2% or 1% of the total cells in the population.

In a particular embodiment, the population obtained in step a) consists of Tscm cells having a cell surface phenotype as defined above, preferably a cell surface phenotype comprising CD4+ or CD8+, CD45RA+, CD95+, CCR7+ and/or CD62L+, preferably comprising CD4+ or CD8+, CD45RA+, CD95+, CCR7+ and/or CD62L+ and CD3+, more preferably comprising CD4+ or CD8+, CD45RA+, CD95+, CCR7+ and/or CD62L+, CD3+ and CD45RO−.

In another particular embodiments, the population obtained in step a) consists of Tscm cells having a cell surface phenotype as defined above, preferably a cell surface phenotype comprising (i) CD4+ or CD8+, CD45RA+, CD95+, CCR7+ and/or CD62L+, PD1− and TIGIT−, preferably comprising CD4+ or CD8+, CD45RA+, CD95+, CCR7+ and/or CD62L+, CD3+, PD1− and TIGIT−, more preferably comprising CD4+ or CD8+, CD45RA+, CD95+, CCR7+ and/or CD62L+, CD3+, CD45RO−, PD1− and TIGIT−, and (ii) optionally LAG3−, TIM3−, CTLA4− and/or CD160−, preferably LAG3− and/or TIM3−.

Preferably, the population obtained in step a) exhibits a ratio CD4+/CD8+ of at least 0.2. In embodiments wherein the population obtained in step a) exhibits a ratio CD4+/CD8+ lower than 0.2, anti-CD40 antibodies may be added to the population in order to compensate for the lack of CD4 T cell helping signals.

In step a), cells having a specific cell surface phenotype are sorted and recovered from a cell sample. Sorting of the cells having a specific cell surface phenotype may be carried out using any method known in the art. Positive and/or negative selection can be readily accomplished using materials and techniques known in the art. For example, cells expressing a particular cell surface marker(s) can be separated from other cells using monoclonal antibodies that bind to the marker and are coupled to columns or magnetic beads; the separation is readily performed according to standard techniques and/or manufacturer or provider directions. In particular, in step a), cells may be sorted by fluorescence-activated cell sorting (FACS) or by magnetic separation.

The cell sample may be any sample containing T cells and in particular Tscm cells, or cells that can be induced in culture to become Tscm cells. Preferably, the sample is a sample containing Tscm cells. Examples of suitable samples include, but are not limited to, a bone marrow cell sample, a blood cell sample, a fractionated or unfractionated whole blood sample, a fractionated or unfractionated apheresis collection (e.g., a leukapheresis collection), tumor infiltrating lymphocytes, PBMCs, or a T cell population (e.g., a population enriched in T cells from a blood sample or PBMCs).

In a particular embodiment, the cell sample is PBMCs. PBMCs can be isolated from a blood sample by any method known in the art such as by ficoll density gradient centrifugation.

As used herein, the term “isolated” means separated from constituents with which the cells are normally associated with in nature.

In another particular embodiment, the cell sample is a population enriched in T cells from PBMCs or from a blood sample, preferably a population enriched in T cells from PBMCs. T cells can be enriched from PBMCs or from a blood sample by any method known in the art. For example, T cells can be enriched from PBMCs or from a blood sample by depletion of CD14+ cells and/or by sorting using an anti-CD3 antibody and retaining CD3+ cells. Preferably, T cells are enriched from PBMCs or from a blood sample by depletion of CD14+ cells and selection of CD3+ cells.

The method may further comprise providing said cell sample from the subject.

As used herein, the term “subject” or “patient” relates to an animal, preferably a mammal, more preferably a human being.

As described below, the population of cells obtained by the method of the invention may be used to provide adoptive cell therapy, in particular autologous therapy (by infusing cells derived from said Tscm cells back into the same patient) or allogeneic therapy (by infusing cells derived from said Tscm cells into another patient). The cell sample may be thus obtained from a healthy subject, in particular for allogeneic therapy, or from a subject having a disease to be treated with said adoptive cell therapy.

In particular, the subject may have an infection or a cancer for which the specific memory T cell responses are functionally impaired. Preferably, this impaired functionality involves a T cell anergy, in particular an anergy of T cell responses against an antigen of interest, i.e. a tumoral or pathogen antigen, and/or T cell exhaustion characterized in particular by high levels of expression of inhibitory receptors such as PD-1 or TIGIT, and/or any other mechanisms of T-cell functional negative regulation. Thus, in preferred embodiments, the subject has an infection or a cancer and exhibits a T cell anergy, in particular an anergy of T cell responses against an antigen of interest, and/or T cell exhaustion, and/or any other mechanisms of T-cell functional negative regulation. In some particular embodiments, the subject has an infection or a cancer and exhibits a T cell anergy, in particular an anergy of T cell responses against an antigen of interest, and/or T cell exhaustion.

Preferably, the subject has a cancer or a pathogen-caused disease as described below, more preferably a disease caused by a polyomavirus, even more preferably a disease caused by a human polyomavirus.

In preferred embodiments, the subject has progressive multifocal leukoencephalitis (PML), Merkel cell carcinoma or BK virus associated nephropathy, preferably has progressive multifocal leukoencephalitis (PML) or Merkel cell carcinoma, more preferably has progressive multifocal leukoencephalitis.

In step b) of the method of the invention, the population of Tscm cells obtained in step a) are cultured in the presence of antigen-presenting cells loaded with at least one antigen of interest or at least one immunogenic peptide derived from at least one antigen of interest, preferably with at least one immunogenic peptide derived from at least one antigen of interest.

Herein, the terms “peptide”, and “protein” are employed interchangeably and refer to a chain of amino acids linked by peptide bonds, regardless of the number of amino acids forming said chain.

The antigen presenting cells (APCs) used in this step can be any antigen presenting cells suitable for presenting said at least one antigen of interest or at least one immunogenic peptide and activating T cells when a major histocompatibility complex (MHC) receptor on the surface of the APC complexed with a peptide interacts with a TCR on the surface of a T cell. Examples of APCs include, but are not limited to, dendritic cells, monocytes, peripheral blood mononuclear cells (PBMCs), Epstein-Barr virus transformed B-lymphoblastoid cell line cells (EBV-BLCL cells), or artificial antigen presenting cells (AAPCs). Preferably, the APCs used in step b) are selected from the group consisting of dendritic cells, monocytes and PBMCs, and combinations thereof. More preferably, the APCs used in step b) are monocytes or dendritic cells, preferably are monocytes.

APCs used in step b) may be autologous (i.e. obtained from the same subject providing the cell sample, and preferably from the subject to be treated) or may be allogeneic (i.e. obtained from another subject than the subject providing the cell sample, and preferably from another subject than the subject to be treated).

In a preferred embodiment, APCs used in step b) are autologous. The skilled person may use a wide range of known procedures to generate autologous APCs using distinct sources, such as peripheral blood monocytes, naturally occurring DCs or CD34+ hematopoietic precursor cells mobilized from the bone marrow. Preferably, autologous APCs are obtained from peripheral blood monocytes or naturally occurring DCs, more preferably from peripheral blood monocytes.

CD34+ stem cells can be differentiated into dendritic cells by incubating the cells with appropriate cytokines, as is known in the art. For example, human CD34+ hematopoietic stem cells can be differentiated in vitro by culturing the cells with human GM-CSF and TNF-α (see, e.g., Szabolcs, et al. (1995) J. Immunol. 154: 5851-5861). Dendritic cells can be then isolated by fluorescence activated cell sorting (FACS) based on expression of cell surface markers or by any other standard methods.

In particular, the method may further comprise before step b) obtaining said autologous APCs from a cell sample from the subject and loading said autologous APCs with at least one antigen of interest or at least one immunogenic peptide derived from at least one antigen of interest, preferably with at least one immunogenic peptide derived from at least one antigen of interest. The cell sample used to obtain autologous APCs may be identical or different from the cell sample used in step a) but both are obtained from the same subject.

Preferably, the method further comprises before step b) sorting from a cell sample from the subject a population of monocytes cells using CD14+ positive selection and loading said monocytes with at least one antigen of interest or at least one immunogenic peptide derived from at least one antigen of interest, preferably with at least one immunogenic peptide derived from at least one antigen of interest. Preferably, the monocytes are obtained from a PBMC sample from the subject.

Alternatively, before or after antigen-loading, monocytes obtained from the sample, e.g. using CD14+ positive selection, may be cultured in the presence of GM-CSF and IL-4 in order to induce differentiation into dendritic cells. Optionally, between day 5 and day 10 of culture, preferably at day 6, IL-6, IL-1β and TNF-α are added to the culture medium during about 24h in order to induce optimal maturation of dendritic cells.

APCs can be loaded by any antigen-loading methods known by the skilled person. For example, APCs, in particular dendritic cells, monocytes or PBMCs may be loaded by pulsing or incubating APCs with one or more antigens of interest and/or one or more peptides, in particular one or more immunogenic peptides, derived from said one or more antigens of interest, or delivering one or more antigens and/or one or more peptides, in particular one or more immunogenic peptides, derived from said one or more antigens of interest into APCs using viral vectors or mRNA transfection.

In preferred embodiments, APCs are loaded with one or more peptides, in particular one or more immunogenic peptides, derived from said one or more antigens of interest, preferably a pool of overlapping peptides, in particular a pool of overlapping immunogenic peptides, derived from said one or more antigens of interest.

An antigen of interest may be any antigen which may be targeted by the immune system to provide a therapeutic effect. The antigen(s) of interest is(are) easily selected by the skilled person depending on the disease to be treated. In preferred embodiments, the antigen(s) of interest is(are) selected depending on the disease to be treated in the subject providing the cell sample, i.e. a cancer or an infection.

In particular, the antigen(s) of interest may be selected from pathogen antigens or antigens expressed by tumor cells such as tumor-specific antigens (TSA) (i.e. antigens found on tumor cells only and not on healthy cells) or tumor-associated antigens (TAA) (i.e. antigens which have elevated levels on tumor cells but are also expressed at lower levels on healthy cells).

In an embodiment, the antigen(s) of interest are selected from one or more antigens of the cancer. The term “cancer” or “tumor”, as used herein, refers to the presence of cells possessing typical features of cancer-causing cells, such as uncontrolled proliferation, immortality, metastatic potential, rapid growth and proliferation rate, and certain characteristic morphological features. This term refers to any type of malignancy (primary or metastasis) and refers to solid or hematopoietic cancers.

In another embodiment, the antigen(s) of interest are selected from one or more antigens of a pathogen, in particular a virus, bacterium or fungus.

In a preferred embodiment, the antigen(s) of interest are selected from one or more viral antigens, preferably one or more antigens from a human virus. Preferably, the virus is selected from the group consisting of polyomaviruses, human immunodeficiency viruses (HIV), human T-lymphotropic virus (HTLV), hepatitis B virus (HBV), hepatitis C virus (HCV), Herpes viruses and Papillomaviruses. More preferably, the virus is selected from the group consisting of human polyomaviruses, in particular from the polyomavirus John Cunningham (JC), the BK virus (BKV) and the Merkel cell polyomavirus (MCPyV or MCV).

In a particular embodiment, the antigen(s) of interest are selected from antigens of a polyomavirus. For example, peptides presented by APCs may include one or more peptides of a polyomavirus, in particular one or more immunogenic peptides, from VP1, VP2, VP3, Large T, small T proteins and/or from any other protein of said polyomavirus. In particular, peptides presented by APCs may be overlapping peptides covering one or several of these proteins. More particularly, peptides presented by APCs, preferably immunogenic peptides, may include overlapping peptide pools covering the VP1, VP2, and VP3 regions of said polyomavirus.

In a more particular embodiment, the antigen(s) of interest are selected from antigens of the polyomavirus MCPyV. For example, peptides presented by APCs may include one or more MCPyV peptides, in particular one or more MCPyV immunogenic peptides, from VP1, VP2, VP3, Large T, small T proteins and/or from any other protein of MCPyV. In particular, peptides presented by APCs may be overlapping peptides covering one or several of these proteins. More particularly, peptides presented by APCs, preferably immunogenic peptides, may include overlapping peptide pools covering the VP1, VP2, and VP3 regions of MCPyV.

In another more particular embodiment, the antigen(s) of interest are selected from antigens of the polyomavirus BKV. For example, peptides presented by APCs may include one or more BKV peptides, in particular one or more BKV immunogenic peptides, from VP1, VP2, VP3, Large T, small T proteins and/or from any other protein of BKV. In particular, peptides presented by APCs may be overlapping peptides covering one or several of these proteins. More particularly, peptides presented by APCs, preferably immunogenic peptides, may include overlapping peptide pools covering the VP1, VP2, and VP3 regions of BKV.

In another more particular embodiment, the antigen(s) of interest are selected from antigens of the polyomavirus JC. For example, peptides presented by APCs may include one or more JCV peptides, in particular one or more JCV immunogenic peptides, from VP1, VP2, VP3, Large T, small T proteins and/or from any other protein of JCV. In particular, peptides presented by APCs may be overlapping peptides covering one or several of these proteins. More particularly, peptides presented by APCs, preferably immunogenic peptides, may include overlapping peptide pools covering the VP1, VP2, and VP3 regions of JCV. Antigens used to load APCs can be prepared by any method known by the skilled person depending on the nature of said antigens. For example, said antigens may be prepared by chemical synthesis, recombinant expression, from a sample from the subject, in particular from subject's own cancer cells, e.g. using whole tumor lysate, or from cancer cell line lysate.

In step b) of the method of the invention, Tscm cells are cultured in the presence of APCs as described above thereby expanding and differentiating into a population comprising T cells reactive to a particular antigen or set of antigens.

Methods to obtain antigen-specific T cells from a population of Tscm cells using APCs are well known in the art and the skilled person may use any of these known methods.

Typically, the culture is carried out in the presence of IL-15, IL-7 and/or other stimulatory cytokines, preferably recombinant cytokines, such as IL-21. Preferably, the culture step comprise culture supplementation with IL-15 and IL-7, and optionally IL-21. Said supplementation starts preferably within the first seven days of culturing, more preferably between day 2 and day 4 of culture. IL-15 and IL-7 may help to maintain stem cell-like phenotype of the Tscm cells

At the beginning of the culture, the ratio of Tscm cells to APCs may be adjusted in order to be set from 1/1 (number of Tscm/number of APC) to 1/20, preferably from 1/5 to 1/15 and more preferably from 1/9 to 1/11.

The culture of Tscm cells in the presence of APCs may last between 8 to 20 days, preferably between 10 to 18 days, more preferably between 12 to 16 days. In a particular embodiment, the culture of Tscm cells in the presence of APCs lasts 14 days.

Optionally, the cells may be cultured for a longer period of time, preferably in the absence of antigen-loaded APCs. In particular, cells may be cultured after step a) and before step b) in the absence of antigen loaded APCs and/or after step b) and before step c), preferably in the absence of antigen loaded APCs.

In some particular embodiments wherein the subject is affected with a retrovirus such as HIV, the culture may be conducted in the presence of one or several antiretroviral compounds.

Optionally, the method of the invention may further comprise step c) recovering cells obtained in step b), in particular CD8+ and/or CD4+ cells, preferably CD8+ and CD4+ cells.

In particular, in step c), the population of cells obtained in step b) may be sorted in order to select CD8+ cells and optionally CD4+ cells.

In particular embodiments, in step c) of the method of the invention, the population of cells obtained in step b) is sorted in order to select/recover CD8+ cells and CD4+ cells. CD8+ cells and CD4+ cells may be recovered separately or together. In some embodiments, CD8+ cells and CD4+ cells are recovered separately, preferably before to being subsequently mixed. This separation allows to adjust the ratio CD8+/CD4+ in the obtained population of cells.

In preferred embodiments, in step c), the population of cells obtained in step b), i.e. all cells of the culture, is recovered and includes CD8+ cells and CD4+ cells. Recovered cells may also include other cell types, in particular APCs such as monocytes or dendritic cells.

Cells may be recovered by any method known by the skilled person including filtration methods or cell sorting methods as described above.

In particular, the population selected/recovered in step c) may comprise Tscm cells (with a cell surface phenotype comprising CD45RA+ CCR7+), T effector (Teff) cells (with a cell surface phenotype comprising CD45RA+ CCR7−), T central memory (Tcm) cells (with a cell surface phenotype comprising CD45RA− CCR7+), and T effector memory (Tem) cells (with a cell surface phenotype comprising CD45RA− CCR7).

In preferred embodiments, Tscm, Tcm and Tem cells represent up to 90%, preferably from 50% to 90%, of the total cells in the selected/recovered population, allowing further cycles of differentiation in vivo and therefore a prolonged therapeutic effect. Typically, Teff cells may represent from 10% to 50%, preferably from 10% to 20%, of the total cells in the selected/recovered population.

In another aspect, the present invention relates to an isolated population of cells comprising antigen-specific CD8+ T cells, and optionally antigen-specific CD4+ T cells, obtained or obtainable by the method of the invention for obtaining a population of cells comprising antigen-specific T cells. Preferably, the population comprises antigen-specific CD8+ T cells and antigen-specific CD4+ T cells.

All embodiments disclosed above and relating to the method for obtaining a population of cells comprising antigen-specific T cells, are also encompassed in this aspect.

In particular, said population may comprise Tscm cells (with a cell surface phenotype comprising CD45RA+ CCR7+), T effector (Teff) cells (with a cell surface phenotype comprising CD45RA+ CCR7−), T central memory (Tcm) cells (with a cell surface phenotype comprising CD45RA− CCR7+), and T effector memory (Tem) cells (with a cell surface phenotype comprising CD45RA− CCR7).

Preferably, Tscm, Tcm and Tem cells represent up to 90%, preferably from 50% to 90%, of the total cells in the isolated population of the invention. Typically, Teff cells may represent from 10% to 50%, preferably from 10% to 20%, of the total cells in the isolated population of the invention.

Preferably, antigen-specific CD8+ T cells represent from 10% to 90% of the total cells and antigen-specific CD4+ T cells represent from 1% to 90% of the total cells in the isolated population of the invention. In particular, antigen-specific CD8+ T cells may represent from 50% to 90% of the total cells in the isolated population of the invention, and antigen-specific CD4+ T cells represent from 1% to 50% of the total cells in the isolated population of the invention.

In a particular embodiment, the isolated population of cells comprising antigen-specific CD8+ T cells, and optionally antigen-specific CD4+ T cells, preferably comprising antigen-specific CD8+ T cells and antigen-specific CD4+ T cells, is obtained or obtainable by a method comprising
a) sorting from a cell sample from a subject suffering from a cancer or a pathogen-caused disease, in particular a cancer or a pathogen-caused disease for which the specific memory T cell responses are functionally impaired, a population of Tscm cells having a cell surface phenotype comprising CD4+ or CD8+, CD45RA+, CD95+, CCR7+ and/or CD62L+,
b) culturing said population of Tscm cells in the presence of antigen-presenting cells, preferably autologous to the subject, loaded with at least one antigen of interest or at least one peptide, in particular at least one immunogenic peptide, derived from said at least one antigen of interest and, optionally, in the presence of IL-7 and IL-15 or other stimulatory cytokines, and optionally
c) recovering cells obtained in step b), in particular CD8+ and/or CD4+ cells, preferably CD8+ and CD4+ cells.

In step c) CD8+ cells, and optionally CD4+ cells, may be sorted from the population of cells obtained in step b).

The subject may suffer from a cancer. In this case, said at least one antigen of interest may be selected from antigens expressed by tumor cells such as tumor-specific antigens (TSA) or tumor-associated antigens (TAA).

The subject may suffer from a pathogen-caused disease. In this case, said at least one antigen of interest may be selected from antigens of said pathogen.

Preferably, the subject suffers from Progressive Multifocal Leukoencephalitis (PML), Merkel cell carcinoma or BK virus associated nephropathy. For subjects suffering from Merkel cell carcinoma, said at least one antigen of interest may be selected from antigens of the Merkel cell polyomavirus (MCPyV or MCV), in particular from VP1, VP2, VP3, Large T, small T proteins and/or from any other protein of MCPyV. For subjects suffering from BK virus associated nephropathy, said at least one antigen of interest may be selected from antigens of the BK virus (BKV), in particular from VP1, VP2, VP3, Large T, small T proteins and/or from any other protein of BKV.

More preferably, the subject suffers from Progressive Multifocal Leukoencephalitis (PML) and said at least one antigen of interest is selected from antigens of the polyomavirus JC, preferably from VP1, VP2, VP3, Large T, small T proteins and/or from any other protein of BKV. For example, peptides presented by APCs, preferably immunogenic peptides, may include JCV overlapping peptide pools covering the VP1, VP2, and VP3 regions of JCV.

Preferably, the population of Tscm cells sorted in step a) has a cell surface phenotype further comprising PD1−, TIGIT−, LAG3−, TIM3−, CTLA4− and/or CD160−, preferably PD1−, TIGIT−, LAG3− and/or TIM3−, more preferably comprising PD1− and TIGIT−, and optionally LAG3− and/or TIM3−, and even more preferably PD1−, TIGIT−, LAG3− and TIM3−.

Preferably, the population of Tscm cells sorted in step a) has a cell surface phenotype further comprising CD3+, CD45RO−, CXCR3+ and/or CD122+, preferably CD3+ and CD45RO−.

In another aspect, the present invention relates to an in vitro method for obtaining a population of memory stem T-cells (Tscm cells), i.e. a population of highly functional Tscm cells, comprising sorting from a cell sample from a subject a population of Tscm cells having a cell surface phenotype comprising CD4+ or CD8+, CD45RA+, CD95+, CCR7+ and/or CD62L+, PD1− and TIGIT−.

Preferably, the population of Tscm cells has a cell surface phenotype further comprising CD3+, CD45RO−, CXCR3+ and/or CD122+, preferably CD3+ and CD45RO−.

Optionally, the method may further comprises depleting cells expressing one or several other inhibitory receptors such as LAG3, TIM3, CTLA4 or CD160. In particular, the method may further comprise depleting cells expressing LAG3, TIM3, CTLA4 and/or CD160, preferably LAG3 and/or TIM3, more preferably LAG3 and TIM3. In this case, the population of sorted cells may have a cell surface phenotype comprising (i) CD4+ or CD8+, CD45RA+, CD95+, CCR7+ and/or CD62L+, PD1− and TIGIT− and (ii) LAG3−, TIM3−, CTLA4 and/or CD160, preferably LAG3− and/or TIM3−, more preferably LAG3− and TIM3−. Preferably, the population of sorted cells has a cell surface phenotype comprising (i) CD4+ or CD8+, CD45RA+, CD95+, CCR7+ and/or CD62L+, CD3+, CD45RO−, PD1− and TIGIT− and (ii) LAG3−, TIM3−, CTLA4 and/or CD160, preferably LAG3− and/or TIM3−, more preferably LAG3− and TIM3−.

Optionally, the method further comprises amplifying the selected population of Tscm. This step may be carried out by any method well-known by the skilled person such as the culture of said Tscm cells in the presence of feeders such as monocytes (non-loaded monocytes), and suitable cytokines.

All embodiments disclosed above and relating to step a) of the method of the invention for obtaining a population of cells comprising antigen-specific T cells, are also encompassed in this aspect.

In another aspect, the present invention relates to an in vitro method for obtaining a population of memory stem T-cells (Tscm cells), i.e. a population of Tscm cells, comprising sorting from a cell sample from a subject a population of Tscm cells having a cell surface phenotype comprising CD4+ or CD8+, CD45RA+, CD95+, CCR7+ and/or CD62L+, and wherein the subject is suffering from a cancer or a pathogen-caused disease, in particular a cancer or a pathogen-caused disease for which the specific memory T cell responses are functionally impaired.

Preferably, the population of Tscm cells has a cell surface phenotype further comprising CD3+, CD45RO−, CXCR3+ and/or CD122+, preferably CD3+ and CD45RO−.

Preferably, the subject has a disease caused by a human polyomavirus.

More preferably, the subject has Progressive Multifocal Leukoencephalitis (PML), Merkel cell carcinoma or BK virus associated nephropathy.

In preferred embodiments, the subject has Progressive Multifocal Leukoencephalitis (PML).

Optionally, the method may further comprises depleting cells expressing one or several inhibitory receptors such as PD1, TIGIT, LAG3, TIM3, CTLA4 or CD160.

In particular, the method may further comprise depleting cells expressing PD1, and/or TIGIT, and optionally cells expressing LAG3, TIM3, CTLA4 and/or CD160, preferably cells expressing PD1 and TIGIT, and optionally cells expressing LAG3 and/or TIM3. In this case, the population of sorted cells may have a cell surface phenotype comprising (i) CD4+ or CD8+, CD45RA+, CD95+, CCR7+ and/or CD62L+, and (ii) PD1−, TIGIT−, LAG3−, TIM3−, CTLA4 and/or CD160, preferably PD1−, TIGIT−, LAG3− and/or TIM3−, more preferably PD1− and TIGIT−, and optionally LAG3− and/or TIM3−. Preferably, the population of sorted cells has a cell surface phenotype comprising (i) CD4+ or CD8+, CD45RA+, CD95+, CCR7+ and/or CD62L+, CD3+ and CD45RO− and (ii) PD1−, TIGIT−, LAG3−, TIM3−, CTLA4 and/or CD160, preferably PD1−, TIGIT−, LAG3− and/or TIM3−, more preferably PD1− and TIGIT−, and optionally LAG3− and/or TIM3−.

More preferably, the method may further comprise depleting cells expressing PD1, TIGIT LAG3 and TIM3. In this case, the population of sorted cells may have a cell surface phenotype comprising (i) CD4+ or CD8+, CD45RA+, CD95+, CCR7+ and/or CD62L+, and (ii) PD1−, TIGIT−, LAG3− and TIM3−. Preferably, the population of sorted cells has a cell surface phenotype comprising (i) CD4+ or CD8+, CD45RA+, CD95+, CCR7+ and/or CD62L+, CD3+ and CD45RO− and (ii) PD1−, TIGIT−, LAG3− and TIM3−.

Optionally, the method further comprises amplifying the selected population of Tscm. This step may be carried out by any method well-known by the skilled person such as the culture of said Tscm cells in the presence of feeders such as monocytes (non-loaded monocytes), and suitable cytokines.

All embodiments disclosed above and relating to step a) of the method of the invention for obtaining a population of cells comprising antigen-specific T cells, are also encompassed in this aspect.

In a further aspect, the present invention relates to an isolated population of Tscm cells obtained or obtainable by the method of the invention for obtaining a population of Tscm cells.

This population may comprise Tscm cells having a cell surface phenotype comprising
(i) CD4+, CD45RA+, CD95+, CCR7+, PD1− and TIGIT−; and/or
(ii) CD8+, CD45RA+, CD95+, CCR7+, PD1− and TIGIT−; and/or,
(iii) CD4+, CD45RA+, CD95+, CD62L+, PD1− and TIGIT−; and/or,
(iv) CD8+, CD45RA+, CD95+, CD62L+, PD1− and TIGIT−; and/or,
(v) CD4+, CD45RA+, CD95+, CCR7+, CD62L+, PD1− and TIGIT−; and/or,
(vi) CD8+, CD45RA+, CD95+, CCR7+, CD62L+, PD1− and TIGIT−.

Preferably, these Tscm cells have a cell surface phenotype further comprising CD3+ and/or CD45RO−, preferably CD3+ and CD45RO−.

Optionally, these Tscm cells may have a cell surface phenotype further comprising LAG3−, TIM3−, CTLA4 and/or CD160, preferably LAG3− and/or TIM3−, more preferably LAG3− and TIM3−.

Preferably, the population of Tscm cells comprises at least 95%, 96%, 97%, 98% or 99% (of the total cells) of Tscm cells having a cell surface phenotype comprising CD4+ or CD8+, CD45RA+, CD95+, CCR7+ and/or CD62L+, PD1− and TIGIT−, preferably CD4+ or CD8+, CD45RA+, CD95+, CCR7+ and/or CD62L+, CD3+, CD45RO−, PD1− and TIGIT−, and optionally LAG3−, TIM3−, CTLA4 and/or CD160, preferably LAG3− and/or TIM3−, more preferably LAG3− and TIM3−.

More preferably, the population of Tscm cells comprises at least 95% or at least 99% (of the total cells) of Tscm cells having a cell surface phenotype comprising CD4+ or CD8+, CD45RA+, CD95+, CCR7+ and/or CD62L+, PD1− and TIGIT−, preferably CD4+ or CD8+, CD45RA+, CD95+, CCR7+ and/or CD62L+, CD3+, CD45RO−, PD1− and TIGIT−, and optionally LAG3−, TIM3−, CTLA4 and/or CD160, preferably LAG3− and/or TIM3−, more preferably LAG3− and TIM3−.

In preferred embodiments, Tscm cells having a cell surface phenotype comprising CD4+ or CD8+, CD45RA+, CD95+, CCR7+ and/or CD62L+, PD1+ and/or TIGIT+, and optionally LAG3+, TIM3+, CTLA4+ and/or CD160+, preferably LAG3+ and/or TIM3+−, represent less than 5% of the total cells in the population, preferably less than 2% or 1% of the total cells in the population.

In a particular embodiment, the population of Tscm cells consists of Tscm cells having a cell surface phenotype comprising CD4+ or CD8+, CD45RA+, CD95+, CCR7+ and/or CD62L+, PD1− and TIGIT−, preferably CD4+ or CD8+, CD45RA+, CD95+, CCR7+ and/or CD62L+, CD3+, CD45RO−, PD1− and TIGIT−, and optionally LAG3−, TIM3−, CTLA4− and/or CD160−, preferably LAG3− and/or TIM3−, more preferably LAG3− and TIM3−.

All embodiments disclosed above and relating to step a) of the method of the invention for obtaining a population of cells comprising antigen-specific T cells or relating to the method of the invention for obtaining a population of Tscm cells are also encompassed in this aspect.

In a further aspect, the present invention also relates to
an isolated population of cells comprising antigen-specific CD8+ T cells, and optionally antigen-specific CD4+ T cells, of the invention (i.e. obtained or obtainable by the method of the invention for obtaining a population of cells comprising antigen-specific T cells), preferably an isolated population of cells comprising antigen-specific CD8+ T cells and antigen-specific CD4+ T cells of the invention, or
an isolated population of Tscm cells of the invention (i.e. obtained or obtainable by the method of the invention for obtaining a population of Tscm cells),
as a cell therapy medicament.

The present invention also relates to said population for use in a cell-based therapy.

All embodiments disclosed above and relating to the method of the invention for obtaining a population of cells comprising antigen-specific T cells, the isolated population of cells comprising antigen-specific CD8+ T cells, and optionally antigen-specific CD4+ T cells, of the invention, the method of the invention for obtaining a population of Tscm cells, and the isolated population of Tscm cells of the invention are also encompassed in this aspect.

In a further aspect, the present invention relates to a pharmaceutical composition comprising
an isolated population of cells comprising antigen-specific CD8+ T cells, and optionally antigen-specific CD4+ T cells, of the invention (i.e. obtained or obtainable by the method of the invention for obtaining a population of cells comprising antigen-specific T cells), preferably an isolated population of cells comprising antigen-specific CD8+ T cells and antigen-specific CD4+ T cells of the invention, or
an isolated population of Tscm cells of the invention (i.e. obtained or obtainable by the method of the invention for obtaining a population of Tscm cells).

In preferred embodiments, the pharmaceutical composition comprises an isolated population of cells comprising antigen-specific CD8+ T cells, and optionally antigen-specific CD4+ T cells, of the invention (i.e. obtained or obtainable by the method of the invention for obtaining a population of cells comprising antigen-specific T cells). Preferably, the pharmaceutical composition comprises an isolated population of cells comprising antigen-specific CD8+ T cells and antigen-specific CD4+ T cells of the invention.

The pharmaceutical composition is formulated in a pharmaceutically acceptable carrier and/or excipient according to the route of administration.

Preferably, the pharmaceutical composition is formulated in order to be suitable for use in a cell based therapy in a subject in need thereof.

The pharmaceutical composition may be formulated in accordance with standard pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York) known by a person skilled in the art.

Preferably, the pharmaceutical composition is suitable for parenteral administration, preferably intravenous infusion.

Pharmaceutical compositions suitable for such administration may comprise the population of cells of the invention, in combination with one or more pharmaceutically acceptable sterile isotonic aqueous or nonaqueous solutions (e.g., balanced salt solution (BSS)), dispersions, suspensions or emulsions, or sterile powders which may be reconstituted into sterile injectable solutions or dispersions just prior to use, which may contain antioxidants, buffers, bacteriostats, solutes or suspending or thickening agents.

Optionally, the composition comprising cells may be frozen for storage at any temperature appropriate for storage of the cells. For example, the cells may be frozen at about −150° C. or −196° C. Cryogenically frozen cells may be stored in appropriate containers and prepared for storage to reduce rick of cell damage and maximize the likelihood that the cells will survive thawing.

The amount of cells to be administered may be determined by standard procedure well known by those of ordinary skill in the art. Physiological data of the patient (e.g. age, size, and weight) and type and severity of the disease being treated have to be taken into account to determine the appropriate dosage.

The pharmaceutical composition of the invention may be administered as a single dose or in multiple doses. Each unit dosage may contain, for example, from 10⁵ to 7.10⁸ cells, preferably from 7.10⁶ to 7.10⁸ cells.

The pharmaceutical composition of the invention may further comprise additional active compounds such as therapeutic monoclonal antibodies to deplete a lymphocyte subset or to block a receptor involved in immune function such as anti-PD-1 or anti-TIGIT.

The present invention also relates to a pharmaceutical composition of the invention for use in a cell-based therapy in a subject in need thereof. The present invention also relates to a pharmaceutical composition of the invention for use in the treatment of a cancer or a pathogen-caused disease. The present invention also relates to a method for treating a subject suffering from a cancer or a pathogen-caused disease, comprising administering to said subject a therapeutically efficient amount of a pharmaceutical composition of the invention. The present invention also relates to the use of a pharmaceutical composition of the invention for preparing a medicament for treating a cancer or a pathogen-caused disease.

All embodiments disclosed above and relating to the method of the invention for obtaining a population of cells comprising antigen-specific T cells, the isolated population of cells comprising antigen-specific CD8+ T cells, and optionally antigen-specific CD4+ T cells, of the invention, the method of the invention for obtaining a population of Tscm cells, the isolated population of Tscm cells of the invention and the pharmaceutical composition of the invention are also encompassed in this aspect.

As used herein, the term “treatment”, “treat” or “treating” refers to any act intended to ameliorate the health status of patients such as therapy, prevention, prophylaxis and retardation of the disease. In certain embodiments, such term refers to the amelioration or eradication of a disease or symptoms associated with a disease. In other embodiments, this term refers to minimizing the spread or worsening of the disease resulting from the administration of one or more therapeutic agents to a subject with such a disease.

The effective amount may be a therapeutically or prophylactically effective amount. A “therapeutically effective amount” refers to an amount effective, at dosages and for periods of time necessary, to achieve the desired therapeutic or prophylactic result. In particular, this term refers to an amount of the pharmaceutical composition of the invention administered to a patient that is sufficient to provide an immune response against the targeted pathogen or tumor cells. The therapeutically effective amount may vary according to various factors such as the disease to be treated, the physiological condition of the subject to be treated, the severity of the affliction and the administration route. A therapeutically effective amount encompasses an amount in which any toxic or detrimental effects are outweighed by the therapeutically beneficial effects. A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount. Suitable means and measures to determine the therapeutically or prophylactically effective amount are available to the person skilled in the art.

Preferably, the pharmaceutical composition is administered via parenteral route, more preferably intravenous infusion. In some embodiments, in particular for the treatment of localized disease, the administration may be targeted in order to deliver cells in the organ or tissue affected by said disease.

In a particular embodiment, the method of the invention comprises administering from 10³ to 10⁸ cells/kg of body weight, preferably 10⁴ to 10⁸ cells/kg of body weight, more preferably from 10¹ to 10⁷ cells/kg of body weight, of an isolated population of cells of the invention, preferably an isolated population of cells comprising antigen-specific CD8+ T cells, and optionally antigen-specific CD4+ T cells, of the invention to said subject.

More particularly, the method of the invention may comprise administering an isolated population of cells comprising antigen-specific CD8+ T cells, and optionally antigen-specific CD4+ T cells, of the invention, and in particular from 1000 to 10,000,000 antigen-specific CD8+ T cells/kg of body weight, preferably from 5000 to 1,000,000 antigen-specific CD8+ T cells/kg of body weight, more preferably from 5000 to 100,000 antigen-specific CD8+ T cells/kg of body weight. In this case, the dose to be administered may be obtained by quantifying CD8+ T cells present in the population or pharmaceutical composition of the invention.

In some embodiments, the method of the invention may comprise administering an isolated population of cells comprising antigen-specific CD8+ T cells and antigen-specific CD4+ T cells, of the invention. Preferably, from 1000 to 10,000,000 antigen-specific CD8+ T cells/kg of body weight, preferably from 5000 to 1,000,000 antigen-specific CD8+ T cells/kg of body weight, more preferably from 5000 to 100,000 antigen-specific CD8+ T cells/kg of body weight are to be administered, and from 1000 to 10,000,000 antigen-specific CD4+ T cells/kg of body weight of the subject, preferably from 5000 to 1,000,000 antigen-specific CD4+ T cells/kg of body weight of the subject, more preferably from 5000 to 100,000 antigen-specific CD4+ T cells/kg of body weight of the subject, are to be administered. In this case, the dose to be administered may be obtained by quantifying CD8+ T cells and optionally quantifying CD4+ T cells present in the population or pharmaceutical composition of the invention. The pharmaceutical composition may be administered as a bolus or repeatedly. The frequency of administration may be for example every two weeks, every month, every three months or every six months.

The treatment may be an autologous therapy (by administering cells derived from a subject back into the same subject) or allogeneic therapy (by administering cells derived from a subject into another subject). Preferably, the treatment is an autologous therapy.

As mentioned above, the subject to be treated, preferably a human being, is suffering from a cancer or pathogen-caused disease.

The cancer or pathogen-caused disease to be treated may be any infection or cancer, in particular any infection or cancer for which the specific memory T cell responses are functionally impaired.

The pathogen-caused disease may be an infection by a virus, a bacterium or a fungus.

To treat pathogen-caused diseases, the pharmaceutical composition may comprise an isolated population of Tscm cells of the invention (i.e. obtained or obtainable by the method of the invention for obtaining a population of Tscm cells). These Tscm cells are administered in order to increase the pool of Tscm cells and allow the in vivo activation of said cells by contacting in vivo APCs.

Preferably, to treat pathogen-caused diseases, the pharmaceutical composition comprises an isolated population of cells comprising antigen-specific CD8+ T cells, and optionally antigen-specific CD4+ T cells, of the invention, preferably an isolated population of cells comprising antigen-specific CD8+ T cells and antigen-specific CD4+ T cells of the invention. To treat this category of disease, the population of cells comprising antigen-specific CD8+ T cells, and optionally antigen-specific CD4+ T cells, of the invention has been obtained by culturing Tscm cells in the presence of APCs loaded with at least one antigen of the pathogen to be targeted or at least one peptide, in particular immunogenic peptide, derived from said at least one antigen, in order to obtain a population of T cells that are activated to recognize target cells bearing said at least one antigen.

Preferably, the pathogen-caused disease is a viral infection, in particular a chronic viral infection. In an embodiment, the pathogen-caused disease is a viral infection caused by a virus selected from the group consisting of polyomaviruses, preferably human polyomaviruses, human immunodeficiency viruses (HIV), human T-lymphotropic virus (HTLV), hepatitis B virus (HBV), hepatitis C virus (HCV), Herpes viruses and Papillomaviruses. Preferably, the virus is selected from the group consisting of human polyomaviruses, in particular from the polyomavirus John Cunningham (JC), the BK virus (BKV) and the Merkel cell polyomavirus (MCPyV or MCV).

In a particular embodiment, the pathogen is the polyomavirus JC and the pathogen-caused disease is Progressive Multifocal Leukoencephalitis (PML).

In another particular embodiment, the pathogen is the BK virus and the pathogen-caused disease is the BK virus-associated nephropathy.

In another particular embodiment, the pathogen is the Merkel cell polyomavirus and the pathogen-caused disease is the Merkel carcinoma.

In another particular embodiment, the pathogen is selected from the group consisting of human immunodeficiency viruses (HIV), human T-lymphotropic virus (HTLV), hepatitis B virus (HBV), hepatitis C virus (HCV), Herpes viruses and Papillomaviruses and the pathogen-caused disease is a chronic or acute viral infection.

To treat a pathogen-caused disease, the cell-based therapy of the invention may be used alone or in combination with other treatment(s) such as antibiotic treatment, antiviral treatment or antiretroviral treatment.

The disease to be treated may also be a solid cancer or a hematopoietic cancer associated or not associated with oncogenic viruses.

The term “cancer” or “tumor”, as used herein, refers to the presence of cells possessing characteristics typical of cancer-causing cells, such as uncontrolled proliferation, immortality, metastatic potential, rapid growth and proliferation rate, and certain characteristic morphological features. This term refers to any type of malignancy (primary or metastases).

Examples of solid cancers include, but are not limited to, breast, stomach, esophageal, sarcoma, ovarian, endometrium, bladder, cervix uteri, rectum, colon, lung or ORL cancers and paediatric tumors (neuroblastoma, glioblastoma multiforme).

Examples of hematopoietic cancers include, but are not limited to, lymphoma, leukemia, myeloma, seminoma, Hodgkin and malignant hemopathies.

To treat this category of diseases, the pharmaceutical composition may comprise an isolated population of cells comprising antigen-specific CD8+ T cells, and optionally antigen-specific CD4+ T cells, of the invention that has been obtained by culturing Tscm cells in the presence of APCs loaded with at least one antigen expressed by tumor cells such as tumor-specific antigens (TSA) or tumor-associated antigens (TAA), or at least one peptide, in particular immunogenic peptide, derived from said at least one antigen, in order to obtain a population of T cells that are activated to recognize target cells bearing said at least one antigen.

Alternatively, and in particular for disease for which there is no identified specific antigen, the pharmaceutical composition may comprise an isolated population of Tscm cells of the invention (i.e. obtained or obtainable by the method of the invention for obtaining a population of Tscm cells). These Tscm cells are administered in order to increase the pool of Tscm cells and allow the in vivo activation of said cells by contacting in vivo APCs.

To treat a cancer, the cell-based therapy of the invention may be used alone or in combination with other treatment(s) such as chemotherapeutic treatment, surgical treatment and/or radiotherapeutic treatment.

In a particular embodiment, the disease to be treated is a pathogen-caused disease wherein the pathogen is the polyomavirus JC and the disease is Progressive Multifocal Leukoencephalitis (PML). In this embodiment, the population of cells comprising antigen-specific T cells to be administered may be obtained by the method comprising
a) sorting from a cell sample from the subject suffering from PML a population of Tscm cells having a cell surface phenotype comprising CD4+ or CD8+, CD45RA+, CD95+, CCR7+ and/or CD62L+, PD1− and TIGIT−, optionally LAG3−, TIM3−, CTLA4− and/or CD160−, preferably LAG3− and/or TIM3−, more preferably LAG3− and TIM3−,
b) culturing said population of Tscm cells in the presence of antigen-presenting cells loaded with at least one antigen of the polyomavirus JC or at least one peptide, in particular immunogenic peptide, derived from said at least one antigen, and, optionally, in the presence of IL-7 and IL-15 or other stimulatory cytokines, and, optionally
c) recovering cells obtained in step b), in particular CD8+ and/or CD4+ cells, preferably CD8+ and CD4+ cells.

Alternatively, the population of cells comprising antigen-specific T cells to be administered may be obtained by the method comprising
a) sorting from a cell sample from the subject suffering from PML a population of Tscm cells having a cell surface phenotype comprising CD4+ or CD8+, CD45RA+, CD95+, CCR7+ and/or CD62L+,
b) culturing said population of Tscm cells in the presence of antigen-presenting cells loaded with at least one antigen of the polyomavirus JC or at least one peptide, in particular immunogenic peptide, derived from said at least one antigen, and, optionally, in the presence of IL-7 and IL-15 or other stimulatory cytokines, and, optionally
c) recovering cells obtained in step b), in particular CD8+ and/or CD4+ cells, preferably CD8+ and CD4+ cells.

Preferably, in step a), the population of Tscm cells has a cell surface phenotype further comprising PD1−, TIGIT−, LAG3−, TIM3−, CTLA4− and/or CD160−, more preferably PD1−, TIGIT−, LAG3− and/or TIM3−.

Preferably, in step a), the population of Tscm cells has a cell surface phenotype further comprising CD3+ and/or CD45RO−, preferably CD3+ and CD45RO−.

In step c), CD8+ cells, and optionally CD4+ cells, may be sorted from the population of cells obtained in step b).

Preferably, peptides presented by APCs may include one or more JCV peptides, in particular one or more JCV immunogenic peptides, from VP1, VP2, VP3, Large T, small T proteins and/or from any other protein of JCV. In particular, peptides presented by APCs may be overlapping peptides covering one or several of these proteins. More particularly, peptides presented by APCs, preferably immunogenic peptides, may include overlapping peptide pools covering the VP1, VP2, and VP3 regions of JCV.

If the subject is further affected with a retrovirus such as HIV, the culture of step b) may be conducted in the presence of an antiretroviral compound.

### Aspects of the Invention

Various aspects and embodiments of the invention are also described in the clauses No. 1 to 15 listed below:

Clause 1. An in vitro method for obtaining a population of cells comprising antigen-specific T cells comprising
a) sorting from a cell sample from a subject a population of Tscm cells having a cell surface phenotype comprising CD4+ or CD8+, CD45RA+, CD95+, CCR7+ and/or CD62L+, PD1− and TIGIT−,
b) culturing said population of Tscm cells in the presence of antigen-presenting cells loaded with at least one immunogenic peptide derived from at least one antigen of interest and, optionally, in the presence of IL-7 and IL-15 or other stimulatory cytokines, and
c) sorting CD8+ cells, and optionally CD4+ cells, from the population of cells obtained in step b).

Clause 2. The method of clause 1, wherein the population of Tscm cells sorted in step a) has a cell surface phenotype further comprising LAG3−, TIM3−, CTLA4− and/or CD160−, preferably LAG3− and/or TIM3−.

Clause 3. The method of clause 1 or 2, wherein the antigen-presenting cells are dendritic cells, monocytes, peripheral blood mononuclear cells (PBMCs), Epstein-Barr virus transformed B-lymphoblastoid cell line cells (EBV-BLCL cells), or artificial antigen presenting cells (AAPCs).

Clause 4. The method of any of clauses 1 to 3, wherein said at least one antigen of interest is a pathogen antigen, preferably a viral, bacterial or fungal antigen, or an antigen expressed by tumor cells such as tumor-specific antigens (TSA) or tumor-associated antigens (TAA) Clause 5. The method of any of clauses 1 to 4, wherein said at least one antigen of interest is an antigen of a human polyomavirus, preferably selected from the group consisting of the polyomavirus JC, the polyomavirus MPCyV or the polyomavirus BK.

Clause 6. The method of any of clauses 1 to 5, wherein said at least one antigen of interest is an antigen of the polyomavirus JC.

Clause 7. An isolated population of cells comprising antigen-specific CD8+ T cells, and optionally antigen-specific CD4+ T cells, obtained or obtainable by the method of any of clauses 1 to 6.

Clause 8. The isolated population of cells of clause 7 comprising, or consisting of, Tscm cells, T effector (Teff) cells, T central memory (Tcm) cells, and T effector memory (Tem) cells.

Clause 9. An isolated population of cells of clause 7 or 8 as a cell therapy medicament.

Clause 10. A pharmaceutical composition comprising an isolated population of cells clause 9, and a pharmaceutically acceptable carrier and/or excipient.

Clause 11. An isolated population of cells of any of clauses 7 to 9 or a pharmaceutical composition of clause 10, for use in the treatment of a cancer or a pathogen-caused disease, preferably a disease caused by a human polyomavirus.

Clause 12. The isolated population of cells or pharmaceutical composition for use of clause 11, wherein the pathogen is the polyomavirus JC and the disease is Progressive Multifocal Leukoencephalitis (PML).

Clause 13. The isolated population of cells or pharmaceutical composition for use of clause 11 or 12, wherein said population is autologous to the subject to be treated.

Clause 14. An in vitro method for obtaining a population of memory stem T-cells (Tscm cells) comprising sorting from a cell sample from a subject a population of Tscm cells having a cell surface phenotype comprising CD4+ or CD8+, CD45RA+, CD95+, CCR7+ and/or CD62L+, PD1− and TIGIT−, and optionally LAG3−, TIM3−, CTLA4− and/or CD160−, preferably LAG3− and/or TIM3−.

Clause 15. An isolated population of Tscm cells having a cell surface phenotype comprising (i) CD4+, CD45RA+, CD95+, CCR7+ and/or CD62L+, PD1−, TIGIT−, and optionally LAG3− and/or TIM3−, and/or (ii) CD8+, CD45RA+, CD95+, CCR7+ and/or CD62L+, PD1−, TIGIT−, and optionally LAG3−, TIM3−, CTLA4− and/or CD160−, preferably LAG3− and/or TIM3−.

All the references cited in this description are incorporated by reference in the present application. Others features and advantages of the invention will become clearer in the following examples which are given for purposes of illustration and not by way of limitation.

### EXAMPLES

### Materials and Methods

### PBMC Isolation

100 mL of heparinized blood were used. Blood was obtained from PML patients with different immunological backgrounds including HIV-infection, hematological malignancies and treatment with immunosuppressive biotherapies. Blood was diluted with Nacl 0.9% (v/v) and PBMCs were isolated by ficoll density gradient centrifugation.

### Inhibitory Receptors Phenotyping

One million PBMCs were stained with the following combination: anti-CD3, anti-CD4, anti-CD8, anti-CD45RA, anti-CD45RO, anti-CCR7, anti-CD95, anti-PD1, anti-TIGIT, anti-LAG-3, anti-TIM-3. CD62L may be used in place of CCR7, if needed. Cells were fixed in PBS 1× containing 1% PFA and analyzed by flow cytometry (BD LSR Fortessa). data were analyzed by means of the FlowJo software.

Cell subsets were defined as follows, in both CD3+ CD4+ T-cells and CD3+ CD8+ T cells
Naive T cell: CD45RA+ CD45RO− CCR7+ CD95−
Stem cell memory (Tscm): CD45RA+ CD45RO− CCR7+ CD95+
Central memory (Tcm): CD45RA− CD45RO+ CCR7+ CD95+
Effector memory (Tem): CD45RA− CD45RO+ CCR7− CD95+
PD1, TIGIT, LAG3, TIM3 expression was analyzed in each CD4+ and CD8+ T cell subset.

### Cell Sorting

### T Cell Subset Isolation

PBMCs were washed in Buffer (PBS 1×, EDTA 2 mM SVF 0.5%). Monocytes were isolated by means of anti-CD14 coated magnetic beads. T cells were subsequently isolated on the negatively selected fraction, by means of antibodies-coated magnetic beads allowing depletion of non-CD3+ cells.

T cells were then washed in PBS containing 0.5% SVF. Cell concentration was adjusted a 20 million cells per mL then cells were stained with the following antibodies: anti-CD3, anti-CD4, anti-CD8, anti-CD45RA, anti-CD62L, anti-CD95, anti-PD1, anti-TIGIT, for 15 minutes at 4° C. Cells were washed, filtered on a 0.22 μm filter to remove cell clumps, and processed for cell sorting.

### Gating Strategy

Cells were first gated on forward and side scatters, then on FSC-A and FSC-H to exclude doublets, then gated as follow:
CD4 Tscm negative for inhibitory receptors: CD3+ CD4+ CD8− CD45RA+ CD45RO− CCR7+ CD95+PD1-TIGIT−
CD4 Tscm Positive for inhibitory receptors: CD3+ CD4+ CD8− CD45RA+ CD45RO− CCR7+CD95+ and NOT(PD1− TIGIT−)
CD8 Tscm negative for inhibitory receptors: CD3+ CD4− CD8+ CD45RA+ CD45RO− CCR7+ CD95+PD1-TIGIT−
CD8 Tscm Positive for inhibitory receptors: CD3+ CD4− CD8+ CD45RA+ CD45RO− CCR7+ CD95+ and NOT(PD1− TIGIT−)

Cells were sorted on an SVF-coated tube. Cells were then resuspended in a culture medium.

### Cell Culture

Five million of monocytes were seeded per well, in 2 mL of culture medium. Overlapping 15-mers JCV peptides, with 11 amino acids overlap, spanning the whole sequence of VP1, VP2 and VP3 proteins (final concentration of 10 μg/mL for each pool) were added for 2 hours at 37° C. Monocytes were then washed twice in culture medium. Purified Tscm were centrifuged and resuspended in culture medium. Tscm were cultured with 5 million of peptide-loaded monocytes in 24-well culture plates in a total volume of 6 mL. This was considered as day 0. On day 2, rIL-7 and rIL-15 were added to the culture (10 ng/ml each, final concentration). 4 mL of medium were removed every 3 days and replaced with fresh medium containing 10 ng/mL of Il-7 and IL-15. On day 14, cells were counted.

### CD4 and CD8 Cell Count

Cells were stained with anti-CD3, anti-CD4, and anti-CD8. Percentages of CD4 and CD8 T cells in the live gate were analyzed. We calculated the number of CD4 and CD8 T cells in each well by multiplying the percentages of CD4 or CD8 T cells by the total cell number contained in each well. Fold expansion was calculated by dividing cell count at day 14 by cell count at DO.

### Cytotoxicity Assay

CD14− and CD3-depleted cells were thawed, washed and incubated for 2 hours with JCV peptides pools spanning VP1, VP2 and VP3 proteins (final concentration: 10 μg/mL for each pool). After washing, cells were used as target cells to restimulate cultured Tscm, at a ratio 1 target cell/10 cultured T cells. Cells were cultured overnight.

Cells were then washed and stained with anti-CD3, anti-CD4, anti-CD8, anti-CD45RA, anti-CD45RO, anti-CCR7, anti-CD27, anti-CD95. Cells were then washed and fixed for 20 minutes at +4° C. Cells were washed and permeabilized. Anti-Granzyme B, and anti-perforin antibodies were added for 30 minutes at +4° C. before a final wash and acquisition by Flow cytometry.

### Analysis

Results were analyzed with the FlowJo Software. Statistical analysis and graphs were performed by means of GraphPad Prism software.

### Results

1—CD4 and CD8 Tscm Express Significantly Less Inhibitory Receptors than Tcm or Tem

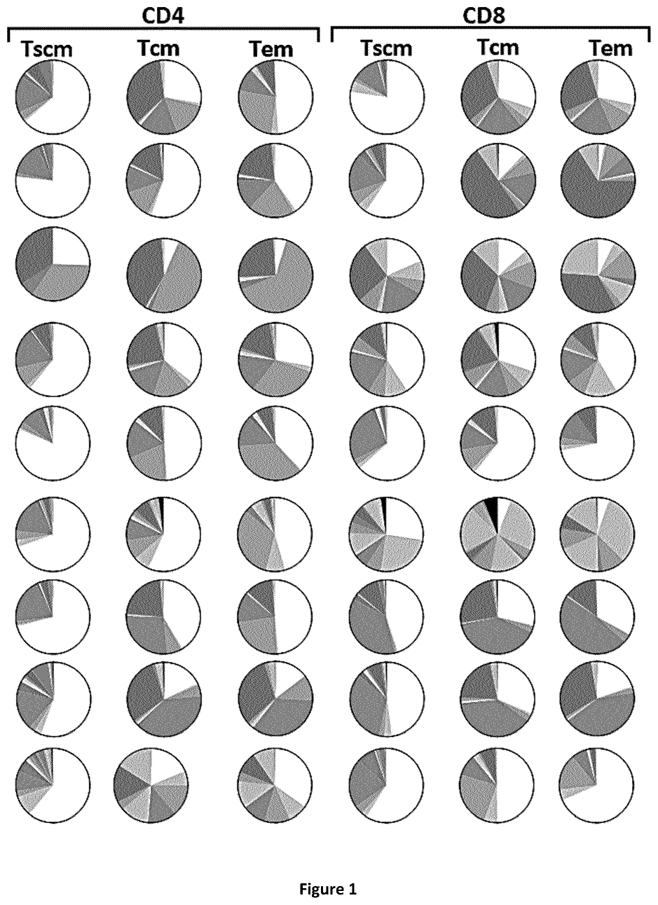

We have analyzed the expression of the inhibitory receptors PD1, TIGIT, LAG3 and TIM3 by Tscm, Tcm and Tem subsets among CD4 and CD8 T cells from PML patients. We have shown in 9 patients that CD4 and CD8 Tscm expressed less inhibitory receptors than Tcm or Tem cells (see FIG. 1: the white part corresponds to cells that express no inhibitory receptors).

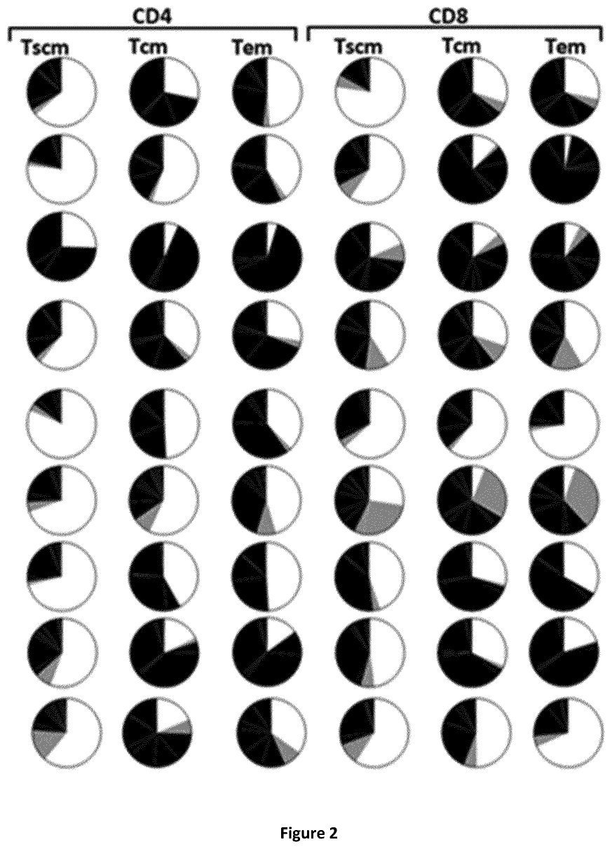

Among these 4 inhibitory receptors, PD1 and TIGIT were the most expressed (FIG. 2, black). The results suggest that a PD1 and TIGIT-based negative selection may enable to deplete a major part of inhibitory receptor-expressing cells. Thus, we isolated Tscm negative for both PD1 and TIGIT, and analyzed their ability to proliferate after in vitro culture.

2—Tscm Negative for PD1 and TIGIT Inhibitory Receptors have Better Cell Proliferation

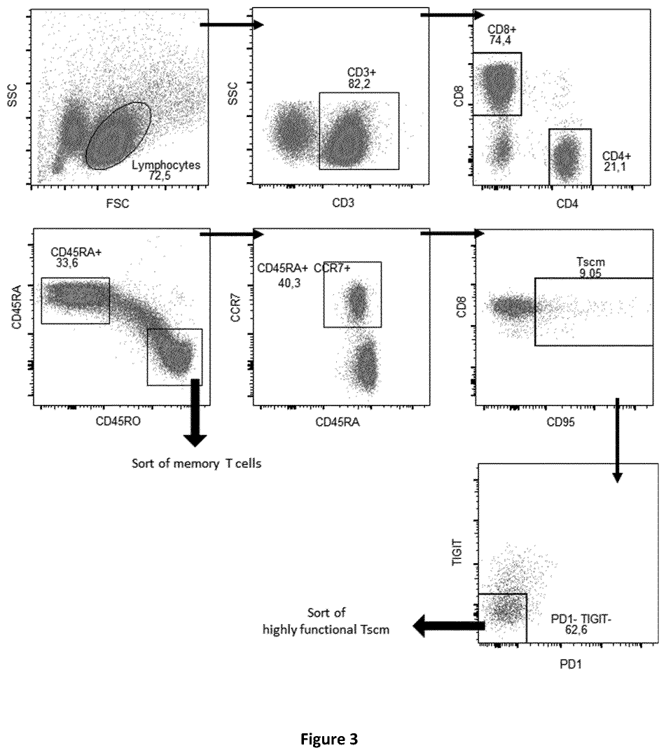

We have established a gating strategy to purify PD1− TIGIT− Tscm cells (see FIG. 3).

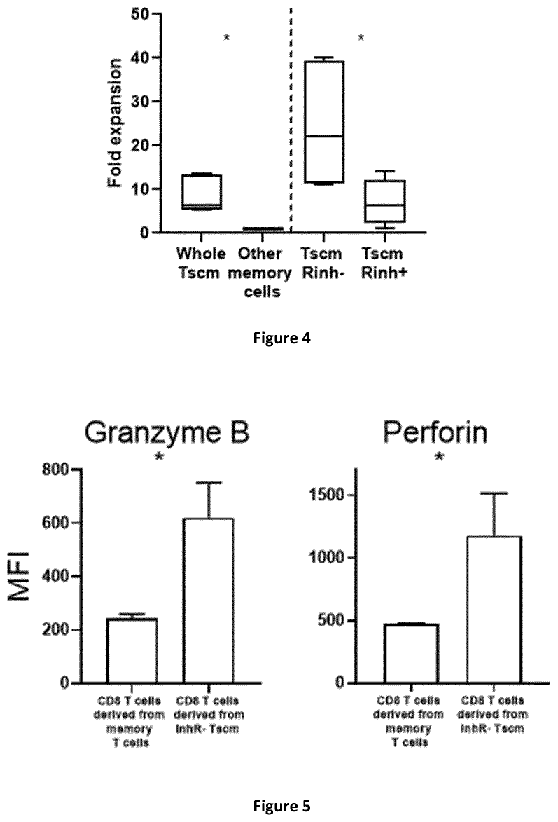

Sorted cells were cultured in the presence of JCV-peptides loaded autologous monocytes for 14 days, in the presence of IL-7 and IL-15, to expand JCV-specific cells. We compared the fold expansion of the cell numbers at the end of the culture, at day 14 (see FIG. 4). We have compared the proliferation ability of i) Tscm versus other memory cells (CD45RO positive cells, including Tcm and Tem, regardless their inhibitory receptors expression status) (see FIG. 4a) and ii) Tscm negative for PD1 and TIGIT vs. Tscm positive for PD1 and/or TIGIT (see FIG. 4b).

These data show that Tscm expand more efficiently than other (more differentiated) memory cells (FIG. 4A), and that PD1− TIGIT− Tscm expand more efficiently than Tscm expressing PD1 and/or TIGIT (FIG. 4B).

### 3—Tscm Negative for PD1 and TIGIT Inhibitory Receptors Show Better Cytotoxic Potential.

Tscm negative for inhibitory receptors were then tested for cytotoxic properties after re-stimulation. At the end of the expansion phase (day 14), JCV-peptide loaded autologous cells were added overnight into the culture. Intracellular perforin and granzyme B were stained. Percentages of CD8 T cells expressing Granzyme B and perforin among total CD8 T cells were determined (see FIG. 5). These data show that effector CD8 T cells derived from Tscm negative for inhibitory receptors display higher cytotoxic potential than Tscm expressing inhibitory receptors or than other (more differentiated) memory CD8 T cells.

4—CD8 T Cells Obtained after In Vitro Culture from Highly Functional Tscm Retain a High Differentiation Capacity that May be Used In Vivo

We analyzed the phenotype of CD8 T cells differentiated either from highly functional stem cell memory (T_{SCM}) negative for PD1 and TIGIT or more differentiated memory T cells (T_{MEM}) at the end of the culture (day 14), based on the following combination:
Stem cell memory (Tscm): CD45RA+ CCR7+
Effector cells (Teff): CD45RA+ CCR7−
Central memory (Tcm): CD45RA− CCR7+
Effector memory (Tem): CD45RA− CCR7−

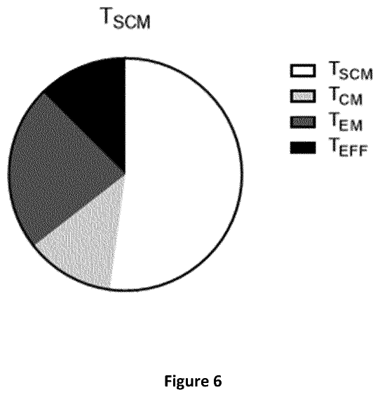

We found that Tscm differentiate into Tcm, Tem and Teff but a large part retains the Tscm phenotype (see FIG. 6).

### CONCLUSION

Altogether, these results demonstrated that:
i) in PML patients from different immunological backgrounds, Tscm cells express lower amounts of inhibitory receptors than more differentiated memory cells. Those inhibitory receptors mostly include PD-1 and TIGIT;
ii) Total Tscm expand better than conventional memory T cells (that include Tcm and Tem);
iii) a selection based on PD1 and TIGIT exclusion allows to deplete a major part of inhibitory receptors expressing Tscm;
iv) Those PD1− TIGIT− Tscm show better proliferation capacities compared to PD1+ and/or TIGIT+ Tscm, and compared to more differentiated memory cells;
v) PD1− TIGIT− Tscm show better cytotoxic capacities compared to PD1+ and/or TIGIT+ Tscm, and compared to more differentiated memory cells; and
vi) PD1− TIGIT− Tscm differentiate efficiently in vitro to more differentiated memory cells including effector cells but a large part retain the Tscm phenotype. This may allow further cycles of differentiation in vivo and therefore a prolonged therapeutic effect.

Altogether, these data show that PD1− TIGIT− Tscm are able to generate an effective and sustained immune response against JCV in PML patients.

1-46. (canceled)

47. An in vitro method for obtaining a population of cells comprising antigen-specific T cells comprising
a) sorting from a cell sample from a subject suffering from a cancer or a pathogen-caused disease, a population of memory stem T-cells (Tscm cells) having a cell surface phenotype comprising CD4+ or CD8+, CD45RA+, CCR7+ and/or CD62L+, and CD95+,
b) culturing said population of Tscm cells in the presence of antigen-presenting cells loaded with at least one antigen of interest or at least one peptide derived from at least one antigen of interest and, optionally, in the presence of IL-7 and IL-15 or other stimulatory cytokines, and optionally
c) recovering cells obtained in step b).

48. The method of claim 47, wherein the population of Tscm cells sorted in step a) has a cell surface phenotype further comprising PD1−, TIGIT−, LAG3−, TIM3−, CTLA4− and/or CD160−.

49. The method of claim 47, wherein the population of Tscm cells sorted in step a) has a cell surface phenotype further comprising PD1− and TIGIT−, and optionally LAG3−, TIM3−, CTLA4− and/or CD160−.

50. The method of claim 47, wherein the population of Tscm cells sorted in step a) has a cell surface phenotype further comprising CD3+, CD45RO−, CXCR3+ and/or CD122+.

51. The method of claim 47, wherein the population of Tscm cells sorted in step a) comprises cells having a cell surface phenotype comprising CD4+, CD8−, CD45RA+, CD95+, CCR7+, PD1− and TIGIT− and cells having a cell surface phenotype comprising CD4−, CD8+, CD45RA+, CD95+, CCR7+, PD1− and TIGIT−.

52. The method of claim 47, wherein the antigen-presenting cells are dendritic cells, monocytes, peripheral blood mononuclear cells (PBMCs), Epstein-Barr virus transformed B-lymphoblastoid cell line cells (EBV-BLCL cells), or artificial antigen presenting cells (AAPCs).

53. The method of claim 47, wherein the antigen-presenting cells are autologous to the subject.

54. The method of claim 47, wherein said at least one antigen of interest is a viral, bacterial or fungal antigen, or an antigen expressed by tumor cells.

55. The method of claim 47, wherein the subject is suffering from a cancer.

56. The method of claim 47, wherein the subject is suffering from a disease caused by a human polyomavirus or has a disease selected from Progressive Multifocal Leukoencephalitis, Merkel cell carcinoma or BK virus associated nephropathy.

57. The method of claim 47, wherein the subject is suffering from Progressive Multifocal Leukoencephalitis and said at least one antigen of interest is an antigen of the polyomavirus JC.

58. The method of claim 47, wherein, in step b), said population of Tscm cells are cultured in the presence of IL-7 and IL-15.

59. The method of claim 47, wherein, in step b), said population of Tscm cells are cultured for 8 to 20 days.

60. The method of claim 47, wherein the cell sample is a bone marrow cell sample, a blood cell sample, a fractionated or unfractionated whole blood sample, a fractionated or unfractionated apheresis collection, tumor infiltrating lymphocytes, PBMCs, or a population enriched in T cells from a blood sample or PBMCs.

61. An isolated population of cells comprising antigen-specific CD8+ T cells, and optionally antigen-specific CD4+ T cells, produced by the method of claim 47.

62. A pharmaceutical composition comprising an isolated population of cells of claim 61, and a pharmaceutically acceptable carrier and/or excipient.

63. A method for treating a subject suffering from a cancer or a pathogen-caused disease, comprising administering to said subject a therapeutically efficient amount of an isolated population of cells of claim 61 or a pharmaceutical composition comprising said isolated population of cells.

64. The method of claim 63, wherein the subject is suffering from a disease caused by a human polyomavirus, Progressive Multifocal Leukoencephalitis, Merkel cell carcinoma or BK virus associated nephropathy.

65. The method of claim 63, wherein the subject is suffering from Progressive Multifocal Leukoencephalitis.

66. An in vitro method for obtaining a population of memory stem T-cells (Tscm cells) comprising sorting from a cell sample from a subject a population of Tscm cells having a cell surface phenotype comprising CD4+ or CD8+, CD45RA+, CD95+, CCR7+ and/or CD62L+, PD1− and TIGIT−, and optionally LAG3−, TIM3−, CTLA4− and/or CD160−.

67. An isolated population of Tscm cells having a cell surface phenotype comprising (i) CD4+, CD45RA+, CD95+, CCR7+ and/or CD62L+, PD1−, TIGIT−, and optionally LAG3−, TIM3−, CTLA4− and/or CD160− and/or (ii) CD8+, CD45RA+, CD95+, CCR7+ and/or CD62L+, PD1−, TIGIT−, and optionally LAG3−, TIM3−, CTLA4− and/or CD160−.
